SEQUENCE LISTING (4 sequences)

SEQ ID NO: 1 - DNA, 96 bp, synthetic construct
  source 1..96: mol_type = other DNA
aatgatacggcgaccaccgagatctacactctttccctacacgacgctcttccgatctttcctgagactctcctgtgcagcctctggattcacctt

SEQ ID NO: 2 - DNA, 89 bp, synthetic construct
  source 1..89: mol_type = other DNA
aatgatacggcgaccaccgagatctacactctttccctacacgacgctcttccgatcttagagccgaggacactgccgtgtattactgt

SEQ ID NO: 3 - DNA, 102 bp, synthetic construct
  source 1..102: mol_type = other DNA
caagcagaagacggcatacgagatnnnnnnnnnngtgactggagttcagacgtgtgctcttccgatctcgctgctcacggtgaccagtgtaccttggcccca

SEQ ID NO: 4 - DNA, 91 bp, synthetic construct
  source 1..91: mol_type = other DNA
caagcagaagacggcatacgagatnnnnnnnnnngtgactggagttcagacgtgtgctcttccgatctgtcagcttggttcctccgccgaa

(19) United States
(12) Patent Application Publication
(10) Pub. No.: US 2026/0018241 A1
(43) Pub. Date: Jan. 15, 2026

(54) METHODS OF ISOLATING ANTI-LIGANDS

(71) Applicant: BioInvent International AB, Lund (SE)

(72) Inventors: Björn FRENDÉUS, Lund (SE); Jenny MATTSSON, Löddeköpinge (SE); Anne LJUNGARS, Kävlinge (SE)

(21) Appl. No.: 18/874,389

(22) PCT Filed: Jun. 12, 2023
(86) PCT No.: PCT/EP2023/065662

§ 371 Date: Dec. 12, 2024

(30) Foreign Application Priority Data
Jun. 13, 2022 (EP) 22178774.0
May 5, 2023 (EP) 23171934.5

(51) Int. Cl. G16B 15/30; G16B 35/20

(52) U.S. Cl. CPC G16B 15/30; G16B 35/20

(57) ABSTRACT

The present invention relates to improved methods of isolating anti-ligands having binding specificities of interest and, in particular, to methods of isolating anti-ligands from anti-ligand libraries where the anti-ligands are specific for differentially and/or infrequently expressed ligands.

The present invention relates to improved methods of isolating anti-ligands having binding specificities of interest and, in particular, to methods of isolating anti-ligands from anti-ligand libraries where the anti-ligands are specific for differentially and/or infrequently expressed ligands.

Protein or peptide-based libraries are often used for selection of anti-ligand molecules with specificity for certain ligands. Such libraries are constructed so that the protein molecule is, in some manner, physically linked to the genetic information encoding the particular protein molecule. The protein molecule is thus displayed together with its gene.

Commonly used display formats rely on cell or virus host particles to present the protein molecule; and include bacterial display (Francisco et al., 1993) and phage display (Smith, 1985; Smith and Scott, 1993; Winter et al., 1994). Such systems display the potential anti-ligand molecule on the surface of the host particle, whilst the genetic information for the displayed molecule is harboured inside the particle and said methods have been employed successfully for selection of specific protein based anti-ligands.

Other display formats relying on in vitro translation exist; including various forms of ribosome display (Mattheakis et al., 1994; Hanes and Pluckthun, 1997; He and Taussig, 1997) that rely on non-covalent linkage of the genetic information to the protein molecule; and other display formats also relying on in vitro translation, whereby a covalent linkage exists between the genetic information and the potential anti-ligand protein molecule, e.g. the Profusion (Weng et al., 2002) or the Covalent Display Technology (Gao et al., 1997).

The displayed peptide or proteinaceous anti-ligand libraries may be totally randomised, (e.g. when peptide libraries are used), or they may be based on a constant region scaffold structure incorporating a further structure conferring variability.

Scaffold structures often used are based on the antibody heavy and light chain variable domains (McCafferty et al., 1990) but may also be based on other scaffolds such as fibronectin (Jacobsson and Frykberg, 1995; Koide et al., 1998), protein A domains (Stahl et al., 1989), or small stable protein domains e.g. BPTI (Markland et al., 1991). Selection of anti-ligands exhibiting a certain binding specificity, from display libraries, is often performed using so called “biopanning” methods.

The target ligand may be immobilised on a solid surface and specific anti-ligand members of a library are exposed to the immobilised target ligand to enable the anti-ligands of interest to bind to the target ligand. Unbound library members are subsequently washed away and the anti-ligands of interest are retrieved and amplified.

In another selection method, the target ligand(s) binds the specific anti-ligand library members whilst in solution. Bound anti-ligands are then isolated using, for example, a retrievable tag attached to the target ligand. The most commonly used tag is biotin, which permits the complex between target molecule and displayed specific library member to be retrieved using avidin coupled to a solid support e.g. a magnetic bead (Parmley and Smith, 1988).

These methods are used when the target ligand is well known and available in a purified form. Selections against a single target ligand at a time are routine. Selection for several defined target ligands may be performed simultaneously. Target ligands may be one or more of small haptens, proteins, carbohydrates, DNA and lipids.

Selections can also be performed against ligands expressed on the surface of cells. This can be done using endogenous cell lines, primary cells, or cells overexpressing a ligand of interest.

Proteinaceous particles other than the members of the anti-ligand library, e.g. phage expressing antibody fragments, may be “sticky” resulting in the binding and isolation of some non-target specific molecules. Non-specific binding may be minimised by adding certain compounds to the anti-ligand display construct/ligand mixture in order to act as blocking agents to reduce this background binding of non-specific anti-ligands e.g. milk, bovine serum albumin, serum (human/foetal calf), gelatine and for certain (non-cellular) applications, detergent.

A number of washing procedures have been devised to reduce non-specific binding of library members to cells and to aid separation of cells from contaminating and/or non-specifically bound library members. Such methods include washing of cells magnetically fixed in a column (Siegel et al., 1997), in order to minimise shearing forces and to allow rebinding of dissociated phage. Another method of washing cells is by centrifugation in a higher density medium such as Ficoll or Percoll, in order to selectively remove non-specific and low affinity anti-ligands and further spatially separate cells and cell-bound anti-ligands from free-anti-ligands and non-specifically bound anti-ligands (Carlsson et al., 1988; Williams and Sharon, 2002).

Depending on the efficiency of the selection process, several rounds of panning may be required to eliminate or at least sufficiently reduce non-specific anti-ligands to a desirable level (Dower et al., 1991).

For many applications, specific anti-ligands against differentially expressed ligands are of interest. For example, proteins may be differentially expressed on cells or present in different amounts in tissues and samples derived from patients with disease, when compared to those from healthy controls. Such diseases include microbial, viral, or parasitic infections, asthma, chronic inflammatory and autoimmune disorders, cancer, neurological-, cardiovascular-, or gastrointestinal disease. Similarly, the protein composition of body fluids, e.g. plasma, cerebrospinal fluid, urine, semen, saliva and mucous, may differ between patients with disease compared to healthy controls.

Consequently, besides their general applicability as research tools to identify differentially expressed ligands, anti-ligands specific for differentially expressed ligands may be used as tools for use in the diagnosis, prevention and/or treatment of disease.

Advances within the genomics and proteomics fields have indicated the presence of a multitude of as yet undefined differentially expressed molecules, stressing the importance of methods for generation of specific anti-ligands for these potential target ligands. Many of these differentially expressed molecules are expected to be present on cell surfaces and thereby constitute potential targets for targeted therapies using, e.g., specific antibodies which may be conjugated to bioactive (e.g. cytotoxic) agents.

Large and highly diversified anti-ligand display libraries provide methods of isolating anti-ligands with specificity to unknown cellular ligands of carbohydrate, protein, lipid, or combined actions thereof.

Biopanning processes currently available include whole-cell, cell-portion, and cell membrane-based methods that, in principle, permit isolation of display constructs exhibiting anti-ligands specific to cell membrane ligands in their native configuration.

Human and humanised therapeutic antibodies are increasingly used to treat diverse diseases including acute and chronic inflammatory disorders, immunological and central nervous system disorders and cancer. Human therapeutic antibodies are considered the most attractive modalities to treat human disease owing to their fully human nature and associated lack of immunogenicity, optimal ability to engage antibody Fc-dependent host immune effector mechanisms, and their superior in vivo half-life compared to their murine, chimeric and humanised counterparts. Human antibodies are today routinely generated by different technologies including humanised mice and highly diversified phage antibody libraries.

Human antibody libraries are further believed to provide advantages compared to transgenic mice carrying human immunoglobulin genes when selecting for antibodies that bind to receptor epitopes that are structurally conserved between man and mouse, since this category of antibodies is negatively selected for in vivo by mechanisms of self-tolerance. Such conserved regions are of particular therapeutic interest since conserved regions often are functionally-associated (e.g. ligand-binding domains necessary for binding and conferral of ligand/receptor induced cellular responses), and antibodies targeting such conserved epitopes may be screened for in vivo therapeutic activity in syngeneic experimental disease model systems.

Large binder libraries (>10¹⁰ members), which physically link antibody genotype to phenotype (biomolecule specificity) by phage-(Smith et al, 1985; McCafferty et al, 1990), yeast-(Boder and Wittrup, 1997), or ribosome (Ellington and Szostak, 1990) display technology, typically contain antibodies that bind with high (nM) affinity and selectivity to many clinically relevant target biomolecules (Hanes et al, 2000; Soderlind et al, 2000; Rothe et al, 2008). Accordingly, antibodies to diverse biomolecules can be isolated from such libraries by the application of positive selection pressure for binding to defined target biomolecules (e.g., a cell surface receptor) and the application of negative selection pressure for binding to highly homologous non-target biomolecules (e.g., related receptors of the same superfamily) (Winter et al, 1994). Similarly, antibodies to a priori unknown biomolecules that are differentially expressed between target and non-target samples can be isolated from antibody libraries by application of positive and negative selection pressure in the form of complex biomolecule populations (e.g., diseased versus normal cells, blood or tissue).

When combined with clinically predictive, high-throughput, functional screening (Frendeus, 2013; Ljungars et al, 2018) and target deconvolution (Mattsson et al, 2021), this allows phenotypic drug discovery (PDD) of medically relevant antibodies, including first-in-class antibodies specific for new targets (Ljungars et al, 2018; Waldmann et al, 1984; Veitonmaki et al, 2013; Roghanian et al, 2015; Williams et al, 2016) and best-in-class antibodies specific to uniquely functional epitopes on validated targets (Semmrich et al, 2022). A broad applicability of PDD to biologics is further indicated by multiple selection strategies incorporating cells, tissues, and fluids to generate antibodies for PDD (Veitonmaki et al, 2013; Roghanian et al, 2015; Dyer et al, 1989). However, realizing biologic PDD full potential (i.e., functional screening of antibodies to all disease-associated biomolecules) will require significant enhancement and output compared to current target-agnostic methods, which have generated few (101 to 103) antibodies specific to a limited number of highly expressed biomolecules (Ljungars et al, 2018; Williams et al, 2016; de Kruif et al, 1995; Ridgway et al, 1999; Qin et al, 2014; Egloff et al, 2019; Sandercock et al, 2015; Nixon et al, 2019).

Furthermore, therapeutic efficacy is, however, not easily predicted from antibody receptor specificity; antibodies to the same target receptor may vary greatly in therapeutic efficacy independent of their binding affinity (Beers et al., 2008; Cragg and Glennie, 2004) and antibodies against alternative molecular targets may show promising, and sometimes unexpected, therapeutic potential (Beck et al., 2010; Cheson and Leonard, 2008). For example, different CD20 specific antibody clones that bound with similar affinity to the CD20 antigen and carried identical mouse IgG2a constant regions, differ fundamentally in ability to deplete B cells in vivo (Beers et al., 2008; Cragg and Glennie, 2004) and antibodies against other tumor-associated cell surface receptors than CD20 can have significant antitumor activity against B cell cancers (for a review see: Cheson and Leonard, 2008). Thus, in a highly diversified antibody library, the most therapeutically efficacious, potent, and best-tolerated antibodies with respect to any given type of cancer may be specific for either of several different receptors, and identifying the therapeutically optimal antibody clones in a highly diversified library requires functional screening of multiple, and ideally all, library members that are specific for different diseased cell-associated receptors.

The applicant has previously developed two different screening technologies (biopanning methods) enabling the retrieval of antibody clones that bind to different surface receptors that are differentially expressed on one cell population (target cells) compared to another (non-target cells) from human phage antibody libraries (hereinafter known as differential biopanning). The first of these methods was described in WO2004/023140 (and also Fransson et al., 2006; Frendeus, 2006). The second of these methods was described in WO2013/041643. The disclosures of WO2004/023140 and WO2013/041643 (and all national filings deriving therefrom) is incorporated by reference herein in their entirety.

The process of WO2004/023140 comprised steps in the following order:
1) differential biopanning, followed by
2) screening for target vs. non-target specificity, followed by
3) conventional sequencing by Sanger technology of a smaller number of clones.

Using this technology, it was possible to generate a pool of antibodies that showed high specificity for target cell versus non-target cell differentially expressed surface receptors.

The process of WO2013/041643 comprised steps in the following order:
1) differential biopanning, followed by
2) high throughput sequencing, followed by
3) confirmatory screening for anti-ligand specificity for the differentially-expressed ligand.

Using the process of WO2004/023140, Sanger sequencing is an example of a technique that is currently used to identify unique binders in a “low throughput” manner. Other examples include running antibody gene DNA on gels before and after restriction enzyme digestion to reveal unique sizes and through different sensitivity to different restriction enzymes, indirectly, different sequences.

When applied to isolating antibodies targeting Cancer B cell (target) versus T cell (non-target) differentially expressed surface receptors (“BnonT” differential biopanning), this process identified antibodies specific for different target cell differentially expressed surface receptors including HLA-DR, surface Ig, and ICAM-1 (Table 1).

TABLE 1
Frequencies and specificities of antibodies isolated by existing
screening methodology, e.g. sequential differential biopanning,
screen for binding, and Sanger sequencing, targeting Cancer
B cell versus T cell differentially expressed surface receptors
(“BnonT” differential biopanning)

| | No of clones | |
|---|---|---|
| Antibody sequence | (out of 81 tested) | Specificity |
| #1 | 71 | sIgM |
| #2 | 4 | HLA-DR |
| #3 | 1 | ICAM-1 |
| #4 | 1 | sIgM |
| #5 | 1 | sIgM |
| #6 | 1 | sIgM |
| #7 | 1 | sIgM |
| #8 | 1 | not determined |

However, targeted receptors were all relatively well expressed, and the number of unique antibody sequences identified (8 out of 81 screened) by this process was limited.

While only a limited number of clones specific for target cell differentially expressed surface receptors were sequenced, the high frequency of one antibody clone indicated limited antibody diversity in the retrieved “BnonT” antibody pool. Thus, while the technology provided a significant improvement compared to previous cell based panning technologies in the sense that antibodies with therapeutic potential to several different differentially expressed receptors were identified by limited screening effort (Fransson et al., 2006), this observation showed that further improvements were required because, in accordance with the prevailing common view, the limited screening for binding can only discover the most common antibodies which are of limited diversity and consist of antibodies against relatively highly expressed and strongly differentially expressed surface receptors (Hoogenboom, 2002) (Liu et al., 2004; Mutuberria et al., 1999; Osbourn et al., 1998).

In silico calculations performed as taught in the earlier biopanning method (WO2004/023140 and Frendeus, 2006) indicated, that the differentially selected “BnonT” antibody pool should contain a much greater number of antibodies against each of several differentially expressed surface receptors.

The sequencing capabilities at that time made sequencing of a significantly greater number of antibody clones in the pool extremely difficult (practically infeasible), therefore the hypothesis that the differentially selected antibody pool should be much more diversified than apparent by the initial screenings was tested using an indirect approach. Thus, using immunobeads conjugated with recombinant ICAM-1 protein (ICAM-1 being a cell surface receptor targeted by a single antibody clone out of the initially 81 sequenced clones in the differentially selected antibody pool of Table 1), the differentially selected “BnonT” antibody pool was panned for the presence of additional ICAM-1 specific antibody clones. Screening of 1260 antibody clones, retrieved following panning of the differentially selected antibody pool against recombinant ICAM-1, identified twenty-one (21) additional ICAM-1 specific antibody sequences/clones.

These observations demonstrated that the original differential biopanning method of WO2004/023140 could identify antibody clones to differentially expressed antigens but that the differentially selected antibody pool was much more diversified than was apparent from these initial screenings, and significantly more so than as determined by conventional screening approaches

The process of WO2013/041643 improved the accuracy of the original differential biopanning method for detecting a plurality of different anti-ligands to a ligand of interest. This improved biopanning method enabled the retrieval of a pool of high affinity anti-ligands such as human antibodies that were specific for different ligands (e.g. receptors) differentially expressed in their native cell surface configuration at low to high levels in a target cell population compared to another cell population(s), from human antibody libraries (and other molecular libraries). This method combined differential biopanning methodology with next generation deep sequencing and used a confirmatory screening for anti-ligand specificity for target cell differentially expressed surface receptors, in effect a “reverse screening”.

Importantly, anti-ligands, such as antibody clones, identified by this approach may all have therapeutic potential because based on firstly, their high affinity binding to receptors that are a) differentially expressed on target cells versus non-target cells and b) expressed in their native cell surface configuration on target cells and secondly the documented ability of antibodies with these properties to mediate therapeutic effects in relevant in vitro and in vivo experimental disease model systems (Beck et al., 2010; Fransson et al., 2006).

Despite the advancements made in the applicant's previous methods, there still remains a need to further improve methods to identify antibodies that are specific to ligands which are differentially expressed between two or more samples, cell types, tissues, fluids etc. Depending on relative and absolute expression level in target and non-target complex biomolecule samples (as discussed above), such anti-ligands may have distinct therapeutic and/or diagnostic value.

The present application addresses that problem by way of the aspects of the invention described below. Specifically, the present application relates to methods that enable, through the generation of predicted and experimental antibody enrichment signatures, a means for preferential identification of anti-ligands to ligands based on ligands expression levels in target and non-target complex biomolecule/ligand samples. This allows identification, expression, and focused analyses of the most promising clones, based on their targeted ligands expression in target and non-target complex biomolecule/ligand samples, in the most appropriate functional assays, which are often restricted with respect to throughput due to scarcity of the biological sample of interest (e.g. primary patient-derived cells). For example, diagnostic antibodies and therapeutic antibodies that rely purely on blockade of ligand-receptor signaling (e.g., anti-IL-6R (31)) could be specific to receptors expressed over a wide dynamic range.

In contrast, Fc-dependent and empowered therapeutic antibodies mediating ADCC and CAR-T cell specificity, owing to their powerful cytolytic nature, require low (Fc-dependent) or no (empowered) receptor expression on critical normal cells and tissue. By enabling identification of anti-ligands that are specific to ligands expressed throughout therapeutically and diagnostically relevant ranges, and, by generating distinct predicted, in silico calculated, and/or experimentally-derived anti-ligand display-selection enrichment profiles, the present invention enables both identification of orders of magnitude greater numbers of anti-ligands compared to existing target agnostic methods, as well as preferential identification of anti-ligands with distinct therapeutic and/or diagnostic potential for prioritised and focused functional screening and target-deconvolution in lower throughput down-stream assays. Accordingly, the method of the present invention enables retrieval of the diversity of therapeutically useful antibodies enriched and identified in differential biopanning studies, and the efficient retrieval of rare antibody clones found in such studies.

In a first aspect of the invention there is provided a method of isolating from a library of anti-ligands at least one anti-ligand to at least one differentially-expressed target ligand in a target cell, tissue, or sample of interest, wherein the method comprises the steps of:
(a) providing one or more reference enrichment signature for the library of anti-ligands used;
(b) performing one or more round of differential biopanning on the library of anti-ligands to produce anti-ligand pools;
(c) performing high throughput sequencing on the anti-ligand pools produced during step (b), so as to generate a discovery enrichment signature for each anti-ligand in the anti-ligand pools.

In preferred embodiments of the first aspect, the method further comprises:
(d) matching the one or more reference enrichment signature provided in step (a) with a discovery enrichment signature for an anti-ligand generated in step (c), so as to isolate at least one anti-ligand to at least one differentially-expressed target ligand in a target cell, tissue, or sample of interest.

By “enrichment signature” we include the meaning of the pattern of changes in the frequency of a given anti-ligand in an anti-ligand population during successive rounds of biopanning. The changes in the frequency of a given anti-ligand from one round to another is determined based on the full anti-ligand population from the preceding round. The enrichment signature provides a means through which the frequency of an anti-ligand in a given anti-ligand population can be tracked and/or predicted during successive biopanning rounds.

By “reference enrichment signature” we include the meaning of a known enrichment signature for a known anti-ligand. The reference enrichment signature may be for an anti-ligand having a known sequence, for an anti-ligand for a known target ligand, and/or for an anti-ligand for a target ligand with a known expression level in a cell, tissue, or sample of interest. The reference enrichment signature may be derived from one or more previously conducted anti-ligand library selection experiments (e.g. using one or more differential biopanning steps), or generated using in silico calculation and modelling.

By “discovery enrichment signature” we include the meaning of an unknown enrichment signature for a previously unknown anti-ligand. The discovery enrichment signature may be for an anti-ligand having a previously unknown sequence, for an anti-ligand for a previously unknown target ligand, and/or for an anti-ligand for a target ligand with a previously unknown expression level in a cell, tissue, or sample of interest. The discovery enrichment signature may be derived by conducting an anti-ligand library selection experiment (e.g. using one or more differential biopanning steps) followed by sequencing of the generated anti-ligand pools.

Enrichment signature information can be used to identify anti-ligands with specificity for differentially expressed ligands by comparing and matching discovery enrichment signatures to reference enrichment signatures for anti-ligands to known ligand targets and/or for anti-ligands to a ligand target having known expression level in a cell, tissue, or sample of interest. Enrichment signature information can also be used to optimise the parameters of a biopanning experiment in order to obtain anti-ligands against a particular different class of ligand, based on the absolute expression level of a ligand in a target cell, tissue, or sample or the relative expression level of a ligand in a target cell, tissue, or sample versus a non-target cell, tissue, or sample.

By “matching” we include the meaning of pairing a reference enrichment signature with a discovery enrichment signature having a similar profile of changes in anti-ligand frequency in the anti-ligand population during successive rounds of biopanning.

By “tissue” we include the meaning of any of the distinct types of material of which animals are made, comprising cells, extracellular matrices and their components, and cell-secreted substances.

By “sample” we include the meaning of a biological sample or specimen. For example, the biological sample/specimen may be a sample/specimen that is taken from a larger entity (e.g a sample/specimen of blood, urine, tissue, or saliva), a cellular lysate, a microbial culture (e.g. bacterial or fungal culture), or a population of viral particles.

In some embodiments of the method of the first aspect, the method further comprises a step of performing screening for anti-ligand specificity for the differentially-expressed ligand, wherein the screening is carried out by:
(i) performing a screening step to identify target vs. non target binding before step (c); and/or,
(ii) performing a confirmatory screening step for anti-ligand specificity for the differentially-expressed ligand after step (c); and/or,
(iii) performing a confirmatory screening step for anti-ligand specificity for the differentially-expressed ligand after step (d).

In some embodiments of the method of the first aspect, the confirmatory screening step is conducted by a binding analysis using methods such as Flow-cytometry, FMAT, ELISA, MSD or CBA.

In some embodiments of the method of the first aspect, the method of isolating at least one anti-ligand does not include a screening step.

By “the method of isolating at least one anti-ligand does not include a screening step” it is meant that the method of isolating anti-ligands does not include:
before step (b), a screening step to identify target vs. non-target binding; nor,
after step (b), a confirmatory step for screening for anti-ligand specificity for the differentially-expressed ligand; nor,
after step (c), performing a confirmatory screening step for anti-ligand specificity for the differentially-expressed ligand; nor,
after step (d), performing a confirmatory screening step for anti-ligand specificity for the differentially-expressed ligand.

In some embodiments of the method of the first aspect, the one or more reference enrichment signature provided in step (a) is an in silico-derived reference enrichment signature.

By “in silico-derived reference enrichment signature” we include the meaning of a reference enrichment signature that has been generated using a computational method, such as in silico modelling of anti-ligand enrichment during successive biopanning rounds.

In some embodiments of the method of the first aspect, the in silico-derived enrichment signature is generated using an equation derived from the universal law of mass of action. In some embodiments of the method of the first aspect, the in silico-derived enrichment signature is generated using the equation:

$${FA}_{T} = \frac{{rA}_{T}}{( {\sum }_{1}^{n} {rA}_{T} )} \times HR$$

where
FA_{T}=frequency of recovered antibody rA_{T}=number of recovered antibodies

$${\sum }_{1}^{n} {rA}_{T} = Total number of recovered antibodies$$

HR=hit-rate, fraction of antibodies specific for the target cells (experimentally determined).

In some embodiments of the method of the first aspect, the in silico-derived reference enrichment signature is for an anti-ligand to a high expressed ligand of a target cell, tissue, or sample of interest, for an anti-ligand to an intermediary expressed ligand of a target cell, tissue, or sample of interest; or for an anti-ligand to a low expressed ligand of a target cell, tissue, or sample of interest.

In some embodiments of the method of the first aspect, the one or more reference enrichment signature provided in step (a) is an experimentally-derived reference enrichment signature. In some embodiments of the method of the first aspect, the experimentally-derived reference enrichment signature is generated from at least one biopanning experiment comprising at least one reference anti-ligand.

In some embodiments of the method of the first aspect, the experimentally-derived reference enrichment signature is for an anti-ligand to a high expressed ligand of a target cell, tissue, or sample of interest, for an anti-ligand to an intermediary expressed ligand of a target cell, tissue, or sample of interest; or for an anti-ligand to a low expressed ligand of a target cell, tissue, or sample of interest.

Experimentally-derived reference enrichment signatures can be generated from one or more previously conducted anti-ligand library selection experiments (e.g. using one or more differential biopanning steps). Experimentally-derived reference enrichment signatures may relate to reference anti-ligands and such reference anti-ligands can be identified in and isolated from anti-ligand libraries by a number of approaches. For example, a reference anti-ligand may be identified in and isolated from an anti-ligand library using an isolated reference ligand (e.g. a purified recombinant protein), using a cell, tissue, or sample enriched for a ligand of interest (e.g. a cell-line engineered to overexpress a protein of interest or a cell line or tissue transformed/transfected with a construct expressing a protein of interest), or using a differential biopanning protocol without prior knowledge of a particular ligand target. Accordingly, it is not always necessary to know the identity of the ligand targeted by a reference anti-ligand, instead it is often sufficient to know only the expression level of the target ligand in target and non-target cells/tissues/samples.

By “reference anti-ligand” we include the meaning of an anti-ligand with a known sequence and/or specificity whose frequency can be tracked in an anti-ligand pool during successive biopanning rounds. Presence of such a reference anti-ligand in an anti-ligand pool can be confirmed using, for instance, nucleic acid sequencing (e.g. next generation sequencing).

By “isolated reference ligand” we include the meaning of an isolated ligand which has known expression level in a cell, tissue, or sample of interest (i.e. in terms of copy number per cell/tissue/sample). An isolated reference ligand is substantially pure and separated from its usual cellular and/or physiological context. For example, an isolated reference ligand can be a purified recombinant protein of interest that is immobilised on affinity beads or a solid surface.

In some embodiments of the method of the first aspect, two or more reference enrichment signatures are provided in step (a), wherein one or more of the reference enrichment signatures is an in silico-derived reference enrichment signature, and wherein one or more of the reference enrichment signatures is an experimentally-derived enrichment signature.

In some embodiments of the method of the first aspect, where two or more reference enrichment signatures are provided in step (a), the one or more in silico-derived enrichment signature is generated using an equation derived from the universal law of mass of action. In some embodiments of the method of the first aspect, where two or more reference enrichment signatures are provided in step (a), the one or more in silico-derived enrichment signature is generated using the equation:

$${FA}_{T} = \frac{{rA}_{T}}{( {\sum }_{1}^{n} {rA}_{T} )} \times HR$$

where
FA_{T}=frequency of recovered antibody
rA_{T}=number of recovered antibodies

$${\sum }_{1}^{n} {rA}_{T} = Total number of recovered antibodies$$

HR=hit-rate, fraction of antibodies specific for the target cells (experimentally determined).

In some embodiments of the method of the first aspect, where two or more reference enrichment signatures are provided in step (a), the one or more in silico-derived reference enrichment signature is for an anti-ligand to a high expressed ligand of a target cell, tissue, or sample of interest, for an anti-ligand to an intermediary expressed ligand of a target cell, tissue, or sample of interest; or for an anti-ligand to a low expressed ligand of a target cell, tissue, or sample of interest.

In some embodiments of the method of the first aspect, where two or more reference enrichment signatures are provided in step (a), the experimentally-derived reference enrichment signature is generated from at least one biopanning experiment comprising at least one reference anti-ligand.

In some embodiments of the method of the first aspect, where two or more reference enrichment signatures are provided in step (a), the one or more experimentally-derived reference enrichment signature is for an anti-ligand to a high expressed ligand of a target cell, tissue, or sample of interest, for an anti-ligand to an intermediary expressed ligand of a target cell, tissue, or sample of interest; or for an anti-ligand to a low expressed ligand of a target cell, tissue, or sample of interest.

In some embodiments of the method of the first aspect, the biopanning step (a) comprises the sub-steps of:
(i) providing a library of anti-ligands;
(ii) providing a first population of ligands comprising a ligand fixed to or incorporated in a subtractor ligand construct;
(iii) providing a second population of ligands comprising a ligand fixed to or incorporated in a target ligand construct;
(iv) determining an amount of the subtractor ligand construct and the target ligand construct in the populations using one or more equations derived from the universal law of mass action

$$\frac{{{[ C ]}^{c} [ D ]}^{d}}{{{[ A ]}^{a} [ B ]}^{b}} = {K}_{eq} ,$$

where:
A, B, C & D=are the participants in the reaction (reactants and products)
a, b, c, & d=the coefficients necessary for a balanced chemical equation
so as to permit isolation of anti-ligand to differentially expressed target ligand;
(v) providing the amount of subtractor ligand construct as determined in step (iv);
(vi) providing the amount of target ligand construct as determined in step (iv);
(vii) providing separation means for isolating anti-ligand bound to the target ligand construct from anti-ligand bound to a subtractor ligand construct;
(viii) exposing the library of (i) to the ligand constructs provided by (v) and (vi) to permit binding of anti-ligands to ligands; and
(ix) using the separation means to isolate anti-ligand bound to the ligand fixed to or incorporated in the target ligand construct.

It is not intended that the steps of the invention necessarily have to be performed in any specific order.

By “providing the determined amount” we include the meaning of providing an amount of ligand that was already known such that the equations of the invention have been used to verify that the known amount provided is suitable for isolating the desired anti-ligand(s).

The reaction parameters that are utilised for a given selection process may be optimised according to the present invention by calculations applying the Mass Law of Action and equations derived therefrom, and taking parameters such as molecular library diversity, anti-ligand copy number, desired detection limit of upregulation, desired anti-ligand affinity, and ligand concentration into consideration.

In some embodiments of the method of the first aspect, the equation in sub-step (iv) is either:

$$bA = \frac{A + B + {K}_{d} {N}_{A} V}{2} - \sqrt{\frac{{( A + B + {K}_{d} {N}_{A} V )}^{2}}{4} - AB}$$

where
bA=number of receptor bound anti-ligands
A=total number of anti-ligands A
B=total number of target receptors B
K_{d}=Anti-ligand affinity (M)
V=the reaction volume (dm³)
NA=Avogadro's constant (6.022×10²³ particles mole⁻¹)
or

$${bA}_{T} = ( \frac{A + B + {K}_{d} {N}_{A} V}{2} - \sqrt{\frac{{( A + B + {K}_{d} {N}_{A} V )}^{2}}{4} - AB} ) \times ( \frac{{B}_{T} {C}_{T}}{{B}_{T} {C}_{T} + {B}_{N} {C}_{N}} ) \qquad ( 5 )$$

where
bA=number of receptor bound anti-ligands
A=total number of anti-ligands A
B=total number of target receptors B
K_{d}=Anti-ligand affinity (M)
V=the reaction volume (dm³)
NA=Avogadro's constant (6.022×10²³ particles mole⁻¹)
CT=the number of target cells
CN=the number of non-target cells
BT=the number of receptors on CT
BN=the number of receptors on CN.

In some embodiments of the method of the first aspect, step (b) of the method comprises performing two or more rounds of biopanning, three or more rounds of biopanning, or four or more rounds of biopanning. Preferably, step (b) comprises performing two or more rounds of biopanning, more preferably three or more rounds of biopanning, most preferably four or more rounds of biopanning.

In some embodiments of the method of the first aspect, the at least one differentially expressed ligand is:
a high expressed ligand of a target cell, tissue, or sample of interest;
an intermediary expressed ligand of a target cell, tissue, or sample of interest; or,
a low expressed ligand of a target cell, tissue, or sample of interest;

In some embodiments of the method of the first aspect, the high expressed ligand is expressed at greater than 1,000,000 copies per target cell of interest, the intermediary expressed ligand is expressed at between 100,000 and 1,000,000 copies per target cell of interest, and the low expressed ligand is expressed at less than 100,000 copies per target cell of interest.

In some embodiments of the method of the first aspect, the ligand is not expressed on one of either the target construct or the subtractor construct.

In some embodiments of the method of the first aspect, the method may further comprise a step of releasing the anti-ligand from the ligand.

In some embodiments of the method of the first aspect, sub-steps (ii) to (ix) are conducted in parallel to isolate a plurality of anti-ligands to a plurality of different ligands. In some embodiment, sub-steps (ii) to (ix) are repeated one or more times.

In some embodiments of the method of the first aspect, the amount of one of the subtractor construct or target construct is provided in excess of the amount of the other of the subtractor construct or target construct. In certain embodiments, the excess of ligand is between 10 and 1000 fold, or 2 and 10 fold, or 1000 and 1,000,000 fold.

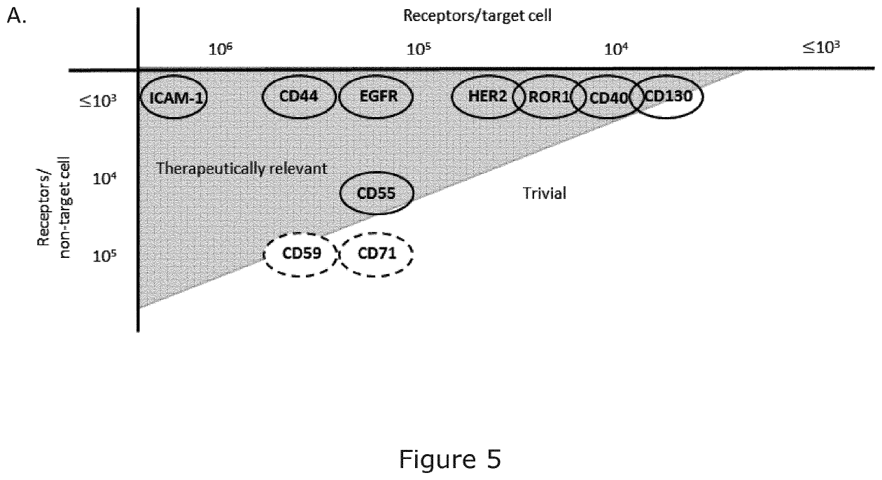

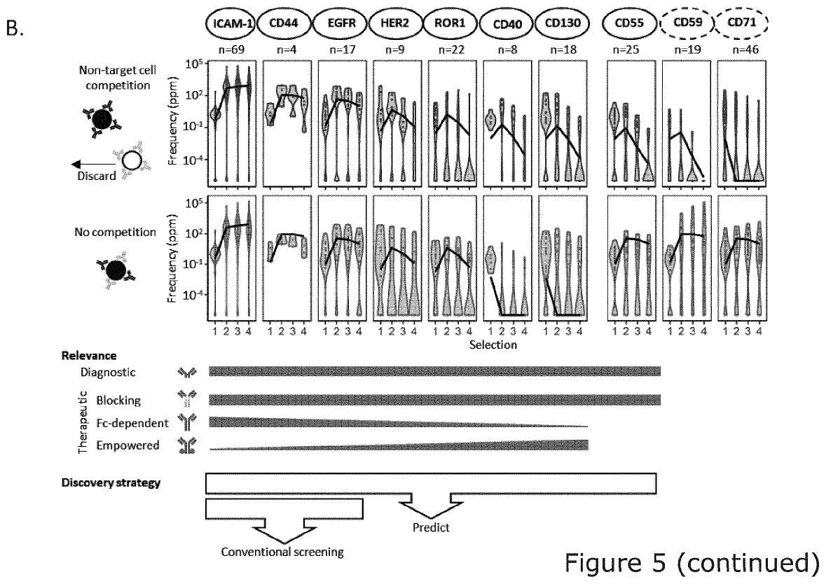

The magnitude of excess of subtractor ligand population determines the highest possible “resolution” (i.e. how well you are able to discriminate between anti-ligands with specificity for ligands that are low upregulated, moderately upregulated, highly upregulated, or uniquely expressed) that you will be able to detect, and how well you will be able to discriminate between differently expressed ligands. For example, if you are using a library with 100 target ligand specific anti-ligands and you add large enough concentrations of positive ligand so that all anti-ligand will be bound to ligand at equilibrium, then a subtractor ligand population excess of 10-fold will allow you to reduce the frequency of anti-ligands with specificity for commonly expressed ligands by 90%, whereas a 200-fold excess (twice the number of anti-ligand specific binders) would allow you to remove common binders (see WO 2004/023140: FIG. 5 and the very last paragraph of example 4 of that document for data confirming this).

In some embodiments of the method of the first aspect, the high throughput sequencing step (c) is carried out using 454 sequencing, Illumina, SOLID methods or the Helicos system, or those from Complete Genomics and Pacific Biosciences.

The advent of next generation sequencing has enabled sequencing of large numbers (1,000s to 1,000,000s) candidate genes in high-throughput manner (from here on referred to as “deep sequencing”)
454 sequencing is described (and incorporated by reference herein) by Margulies et al. (2005). In the 454 method, the DNA to be sequenced is either fractionated and supplied with adaptors or segments of DNA can be PCR-amplified using primers containing the adaptors. The adaptors are nucleotide 25-mers required for binding to the DNA Capture Beads and for annealing the emulsion PCR Amplification Primers and the Sequencing Primer. The DNA fragments are made single stranded and are attached to DNA capture beads in a manner that allows only one DNA fragment to be attached to one bead. Next, the DNA containing beads are emulsified in a water-in-oil mixture resulting in microreactors containing just one bead.

Within the microreactor, the fragment is PCR-amplified, resulting in a copy number of several million per bead. After PCR, the emulsion is broken and the beads are loaded onto a pico titer plate. Each well of the pico-titer plate can contain only one bead. Sequencing enzymes are added to the wells and nucleotides are flowed across the wells in a fixed order. The incorporation of a nucleotide results in the release of a pyrophosphate, which catalyses a reaction leading to a chemiluminescent signal. This signal is recorded by a CCD camera and a software is used to translate the signals into a DNA sequence.

In the Illumina method (Bentley (2008)), single stranded, adaptor-supplied fragments are attached to an optically transparent surface and subjected to “bridge amplification”. This procedure results in several million clusters, each containing copies of a unique DNA fragment. DNA polymerase, primers and four labelled reversible terminator nucleotides are added and the surface is imaged by laser fluorescence to determine the location and nature of the labels. Protecting groups are then removed and the process is repeated for several cycles.

The SOLID process (Shendure (2005)) is similar to 454 sequencing, DNA fragments are amplified on the surface of beads. Sequencing involves cycles of ligation and detection of labelled probes.

Several other techniques for high-throughput sequencing are currently being developed. Examples of such are The Helicos system (Harris (2008)), Complete Genomics (Drmanac (2010)) and Pacific Biosciences (Lundquist (2008)). As this is an extremely rapidly developing technical field, the applicability to the present invention of high throughput sequencing methods will be obvious to a person skilled in the art

In some embodiments of the method of the first aspect, the separation means is selected from at least one of a solid support, cell membrane and/or portions thereof, synthetic membrane, beads, chemical tags and free ligand, or fluorescence activated cell sorting.

In some embodiments of the method of the first aspect, sub-step (ix) may be performed by at least one of density centrifugation (Williams and Sharon, 2002), solid support sequestration, magnetic bead sequestration using beads specific for receptors expressed on one cell population or biotin-specific beads after biotinylation of one cell population (Siegel et al., 1997), chemical tag binding and aqueous phase partitioning, fluorescent activated cell sorting.

In some embodiments of the method of the first aspect, the library of anti-ligands is a display library comprising a plurality of library members which display anti-ligands. In some embodiments of the method of the first aspect, the anti-ligand display library comprising a plurality of library members which display anti-ligands is a phage display library, an RNA display library, a ribosome display library, a yeast display library, or a mammalian display library. Preferably, the library of anti-ligands is a phage display library.

In some embodiments of the method of the first aspect, the library of anti-ligands is constructed from at least one of antibodies, antigen binding variants, derivatives and/or fragments thereof; scaffold molecules with engineered variable surfaces; receptors; and enzymes. Preferably, the library of anti-ligands is constructed from antibodies, antigen binding variants, derivatives and/or fragments thereof

In some embodiments of the method of the first aspect, the library of anti-ligands comprises at least one anti-ligand having a known copy number. In some embodiments of the method of the first aspect, the at least one anti-ligand having a known copy number is added to the library of anti-ligands at a known copy number. For instance, the library of anti-ligands may contain a sequence encoding a commercially available anti-ligand (e.g. a commercially available antibody) that has been added to the library at a known copy number and can be used to generate an experimentally-derived enrichment signature.

In some embodiments of the method of the first aspect, the library of anti-ligands is a pool of anti-ligands generated by a previously conducted anti-ligand library selection experiment.

The display of proteins and polypeptides on the surface of bacteriophage (phage), fused to one of the phage coat proteins, provides a powerful tool for the selection of specific ligands. This ‘phage display’ technique was originally used by Smith in 1985 to create large libraries of antibodies for the purpose of selecting those with high affinity for a particular antigen. More recently, the method has been employed to present peptides, domains of proteins and intact proteins at the surface of phage in order to identify ligands having desired properties.

The principles behind phage display technology are as follows:
(i) Nucleic acid encoding the protein or polypeptide for display is cloned into a phage;
(ii) The cloned nucleic acid is expressed fused to the coat-anchoring part of one of the phage coat proteins (typically the p3 or p8 coat proteins in the case of filamentous phage), such that the foreign protein or polypeptide is displayed on the surface of the phage;
(iii) The phage displaying the protein or polypeptide with the desired properties is then selected (e.g. by affinity chromatography) thereby providing a genotype (linked to a phenotype) that can be sequenced, multiplied and transferred to other expression systems.

Alternatively, the foreign protein or polypeptide may be expressed using a phagemid vector (i.e. a vector comprising origins of replication derived from a phage and a plasmid) that can be packaged as a single stranded nucleic acid in a bacteriophage coat. When phagemid vectors are employed, a “helper phage” is used to supply the functions of replication and packaging of the phagemid nucleic acid. The resulting phage will express both the wild type coat protein (encoded by the helper phage) and the modified coat protein (encoded by the phagemid), whereas only the modified coat protein is expressed when a phage vector is used.

The use of phage display to isolate ligands that bind biologically relevant molecules has been reviewed in Felici et al. (1995), Katz (1997) and Hoogenboom et al. (1998). Several randomised combinatorial peptide libraries have been constructed to select for polypeptides that bind different targets, e.g. cell surface receptors or DNA (Kay and Paul, (1996)).

Proteins and multimeric proteins have been successfully phage-displayed as functional molecules (see Chiswell and McCafferty, (1992)). In addition, functional antibody fragments (e.g. Fab, single chain Fv [scFv]) have been expressed (McCafferty et al. (1990); Barbas et al. (1991); Clackson et al. (1991)), and some of the shortcomings of human monoclonal antibody technology have been superseded since human high affinity antibody fragments have been isolated (Marks et al. (1991) and Hoogenboom and Winter (1992)).

Further information on the principles and practice of phage display is provided in Phage display of peptides and proteins: a laboratory manual Ed Kay, Winter and McCafferty (1996), the disclosure of which is incorporated herein by reference.

The library of anti-ligands can be constructed from at least one selected from antibodies, and antigen binding variants, derivatives or fragments thereof; scaffold molecules with engineered variable surfaces; receptors; and enzymes.

In some embodiments of the method of the first aspect, the ligand is at least one selected from antigens; receptor ligands; and enzyme targets that comprise at least one of carbohydrate; protein; peptide; lipid; polynucleotide; inorganic molecules and conjugated molecules. Preferably, the ligand is a cell surface receptor. More preferably, the cell surface receptor is in its native form.

In some embodiments of the method of the first aspect, the method further comprises a step of exposing the ligand and its separation means to a stimulus which influences the expression of target ligands on said ligand constructs.

In a second aspect of the invention there is provided a method of isolating from a library of anti-ligands at least one anti-ligand to at least one differentially-expressed target ligand in a target cell, tissue, or sample of interest, wherein the method comprises the steps of:
(a) providing one or more reference enrichment signature for an anti-ligand present in the library of anti-ligands used; and,
(b) providing one or more discovery enrichment signature for an anti-ligand present in the library of anti-ligands used.

In preferred embodiments of the second aspect, the method further comprises:
(c) matching the one or more reference enrichment signature provided in step (a) with the one or more discovery enrichment signature provided in step (b), so as to isolate at least one anti-ligand to at least one differentially-expressed target ligand in a target cell, tissue, or sample of interest.

In some embodiments of the method of the second aspect, the method further comprises a step of performing screening for anti-ligand specificity for the differentially-expressed ligand, wherein the screening is carried out by:
(i) performing a confirmatory screening step for anti-ligand specificity for the differentially-expressed ligand after step (b); and/or,
(ii) performing a confirmatory screening step for anti-ligand specificity for the differentially-expressed ligand after step (c).

In some embodiments of the method of the second aspect, the method of isolating at least one anti-ligand does not include a screening step.

By “the method of isolating at least one anti-ligand does not include a screening step” it is meant that the method of isolating anti-ligands does not include:
before step (b), a screening step to identify target vs. non-target binding; nor,
after step (b), a confirmatory step for screening for anti-ligand specificity for the differentially-expressed ligand; nor,
after step (c), performing a confirmatory screening step for anti-ligand specificity for the differentially-expressed ligand; nor,
after step (d), performing a confirmatory screening step for anti-ligand specificity for the differentially-expressed ligand.

In some embodiments of the method of the second aspect, the one or more reference enrichment signature provided in step (a) is an in silico-derived reference enrichment signature.

In some embodiments of the method of the second aspect, the in silico-derived enrichment signature is generated using an equation derived from the universal law of mass of action. In some embodiments of the method of the second aspect, the in silico-derived enrichment signature is generated using the equation:

$${FA}_{T} = \frac{{rA}_{T}}{( {\sum }_{1}^{n} {rA}_{T} )} \times HR$$

where
FA_{T}=frequency of recovered antibody
rA_{T}=number of recovered antibodies

$${\sum }_{1}^{n} {rA}_{T} = Total number of recovered antibodies$$

HR=hit-rate, fraction of antibodies specific for the target cells (experimentally determined).

In some embodiments of the method of the second aspect, the in silico-derived reference enrichment signature is for an anti-ligand to a high expressed ligand of a target cell, tissue, or sample of interest, for an anti-ligand to an intermediary expressed ligand of a target cell, tissue, or sample of interest; or for an anti-ligand to a low expressed ligand of a target cell, tissue, or sample of interest.

In some embodiments of the method of the second aspect, the one or more reference enrichment signature provided in step (a) is an experimentally-derived reference enrichment signature. In some embodiments of the method of the second aspect, the experimentally-derived enrichment signature is generated from at least one biopanning experiment comprising at least one reference ligand.

In some embodiments of the method of the second aspect, the experimentally-derived reference enrichment signature is for an anti-ligand to a high expressed ligand of a target cell, tissue, or sample of interest, for an anti-ligand to an intermediary expressed ligand of a target cell, tissue, or sample of interest; or for an anti-ligand to a low expressed ligand of a target cell, tissue, or sample of interest.

Experimentally-derived reference enrichment signatures can be generated from one or more previously conducted anti-ligand library selection experiments (e.g. using one or more differential biopanning steps). Experimentally-derived reference enrichment signatures may relate to reference anti-ligands and such reference anti-ligands can be identified in and isolated from anti-ligand libraries by a number of approaches. For example, a reference anti-ligand may be identified in and isolated from an anti-ligand library using an isolated reference ligand (e.g. a purified recombinant protein), using a cell, tissue, or sample enriched for a ligand of interest (e.g. a cell-line engineered to overexpress a protein of interest or a cell line or tissue transformed/transfected with a construct expressing a protein of interest), or using a differential biopanning protocol without prior knowledge of a particular ligand target. Accordingly, it is not always necessary to know the identity of the ligand targeted by a reference anti-ligand, instead it is often sufficient to know only the expression level of the target ligand in target and non-target cells/tissues/samples.

In some embodiments of the method of the second aspect, two or more reference enrichment signatures are provided in step (a), wherein one or more of the reference enrichment signatures is an in silico-derived reference enrichment signature, and wherein one or more of the reference enrichment signatures is an experimentally-derived enrichment signature.

In some embodiments of the method of the second aspect, where two or more reference enrichment signatures are provided in step (a), the one or more in silico-derived enrichment signature is generated using an equation derived from the universal law of mass of action. In some embodiments of the method of the first aspect, where two or more reference enrichment signatures are provided in step (a), the in silico-derived enrichment signature is generated using the equation:

$${FA}_{T} = \frac{{rA}_{T}}{( {\sum }_{1}^{n} {rA}_{T} )} \times HR$$

where
FA_{T}=frequency of recovered antibody
rA_{T}=number of recovered antibodies

$${\sum }_{1}^{n} {rA}_{T} = Total number of recovered antibodies$$

HR=hit-rate, fraction of antibodies specific for the target cells (experimentally determined).

In some embodiments of the method of the second aspect, where two or more reference enrichment signatures are provided in step (a), the one or more in silico-derived reference enrichment signature is for an anti-ligand to a high expressed ligand of a target cell, tissue, or sample of interest, for an anti-ligand to an intermediary expressed ligand of a target cell, tissue, or sample of interest; or for an anti-ligand to a low expressed ligand of a target cell, tissue, or sample of interest.

In some embodiments of the method of the second aspect, where two or more reference enrichment signatures are provided in step (a), the experimentally-derived enrichment signature is generated from at least one biopanning experiment comprising at least one reference ligand.

In some embodiments of the method of the second aspect, where two or more reference enrichment signatures are provided in step (a), the experimentally-derived reference enrichment signature is for an anti-ligand to a high expressed ligand of a target cell, tissue, or sample of interest, for an anti-ligand to an intermediary expressed ligand of a target cell, tissue, or sample of interest; or for an anti-ligand to a low expressed ligand of a target cell, tissue, or sample of interest.

In some embodiments of the method of the second aspect, the one or more discovery enrichment signature provided in step (b) is derived from a previously conducted anti-ligand library selection experiment.

In some embodiments of the method of the second aspect, the at least one differentially expressed ligand is:
a high expressed ligand of a target cell, tissue, or sample of interest;
an intermediary expressed ligand of a target cell, tissue, or sample of interest; or,
a low expressed ligand of a target cell, tissue, or sample of interest;

In some embodiments of the method of the second aspect, the high expressed ligand is expressed at greater than 1,000,000 copies per target cell of interest, the intermediary expressed ligand is expressed at between 100,000 and 1,000,000 copies per target cell of interest, and the low expressed ligand is expressed at less than 100,000 copies per target cell of interest.

In some embodiments of the method of the second aspect, the library of anti-ligands is a display library comprising a plurality of library members which display anti-ligands. In some embodiments of the method of the first aspect, the anti-ligand display library comprising a plurality of library members which display anti-ligands is a phage display library, an RNA display library, a ribosome display library, a yeast display library, or a mammalian display library. Preferably, the library of anti-ligands is a phage display library.

In some embodiments of the method of the second aspect, the library of anti-ligands is constructed from at least one of antibodies, antigen binding variants, derivatives and/or fragments thereof; scaffold molecules with engineered variable surfaces; receptors; and enzymes. Preferably, the library of anti-ligands is constructed from antibodies, antigen binding variants, derivatives and/or fragments thereof.

In some embodiments of the method of the first aspect, the library of anti-ligands comprises at least one anti-ligand having a known copy number. In some embodiments of the method of the second aspect, the at least one anti-ligand having a known copy number is added to the library of anti-ligands at a known copy number. For instance, the library of anti-ligands may contain a sequence encoding a commercially available anti-ligand (e.g. a commercially available antibody) that has been added to the library at a known copy number and can be used to generate an experimentally-derived enrichment signature.

In some embodiments of the method of the second aspect, the library of anti-ligands is a pool of anti-ligands generated by a previously conducted anti-ligand library selection experiment.

In some embodiments of the method of the second aspect, the ligand is at least one selected from antigens; receptor ligands; and enzyme targets that comprise at least one of carbohydrate; protein; peptide; lipid; polynucleotide; inorganic molecules and conjugated molecules. Preferably, the ligand is a cell surface receptor. More preferably, the cell surface receptor is in its native form.

In a third aspect of the invention there is provided an enrichment signature for an anti-ligand, wherein the enrichment signature is generated by step (c) of a method according to the first aspect.

Selected anti-ligands identified by the method of the first aspect of the invention may subsequently be used in the manufacture of a pharmaceutical composition for use in medicine for the treatment, imaging, diagnosis or prognosis of disease. Anti-ligands based on antibodies and most importantly on human antibodies have great therapeutic potential.

Therefore, in a fourth aspect of the invention there is provided a method for preparing a pharmaceutical composition which comprises, following the identification of an anti-ligand with desired characteristics by a method according to the first or second aspect, adding said anti-ligand to a pharmaceutically acceptable carrier.

In a fifth aspect of the invention there is provided a pharmaceutical composition prepared by the method according to the fourth aspect.

In a sixth aspect of the invention there is provided a pharmaceutical composition prepared by the method of the fourth aspect for use in medicine. The pharmaceutical composition may also be used in the manufacture of a medicament for the prevention, treatment, imaging, diagnosis or prognosis of disease.

### Other Definitions

By “biopanning” we include the meaning of a method of selection of one member from a desired anti-ligand-ligand-binding pair, based on its ability to bind with high affinity to the other member.

By “differential biopanning” we include the meaning of a biopanning method to select one member from a desired anti-ligand-ligand-binding pair that is expressed in different amounts in or on two different sources (e.g. a subtractor/control and target), based on its ability to bind with high affinity to the other member

By “high throughput sequencing” and “deep sequencing” we include the meaning of a sequencing process in which a large number of sequences are sequenced in parallel (up to millions) such that the speed of sequencing large numbers of molecules is practically feasible and made significantly quicker and cheaper.

By “confirmatory screening” we include the meaning of detecting specific ligand binding of the isolated anti-ligand pool and/or individual anti-ligand clones to a target construct vs. a subtractor construct using any assay addressing ligand/anti-ligand binding (e.g. Flow-cytometry, FMAT, ELISA, MSD and CBA). The term further includes the meaning that once an anti-ligand is identified as binding to a differentially expressed ligand, the nature and identity of the ligand and the binding interactions between anti-ligand and ligand are studied

By “ligand” we include the meaning of one member of a ligand/anti-ligand binding pair. The ligand may be, for example, one of the nucleic acid strands in a complementary, hybridised nucleic acid duplex binding pair; an effector molecule in an effector/receptor binding pair; or an antigen in an antigen/antibody or antigen/antibody fragment binding pair.

By “anti-ligand” we include the meaning of the opposite member of a ligand/anti-ligand binding pair. The anti-ligand may be the other of the nucleic acid strands in a complementary, hybridised nucleic acid duplex binding pair; the receptor molecule in an effector/receptor binding pair; or an antibody or antibody fragment molecule in antigen/antibody or antigen/antibody fragment binding pair, respectively.

By “antigen” we include the meaning of a molecule or chemical compound that is able to interact with antibodies but not necessarily produce an immune response. Such antigens include, but are not limited to molecules of protein, peptide, nucleotide, carbohydrate, lipid or a conjugate thereof.

By “differentially expressed ligands” we include the meaning of those ligands that are either expressed at differing levels between the target and subtractor sources, including those expressed only in certain conditions/places and not in others; or where either the target or subtractor ligand is a modified version of the other from the target and subtractor ligands. For example, some antigens are highly expressed on the cell surfaces of diseased cells (e.g. cancer cells) and at low levels or not at all on the equivalent healthy cells (e.g. non-cancerous cells).

By “low expression ligands” we mean ligands that are expressed at relatively low levels (i.e. less than 100,000 copies per cell/tissue/sample), or ligands occurring at a frequency of less than 1% of any other, more highly expressed ligand in the positive ligand population sample.

By “intermediary expressed ligands” we mean ligands that are expressed at relatively intermediate levels (i.e. from 100,000 copies per cell/tissue/sample to 1,000,000 copies per cell/tissue/sample).

By “high expressed ligands” we mean ligands that are expressed at relatively high levels (i.e. greater than 1,000,000 copies per cell/tissue/sample).

By “ligand construct” we include the meaning of a system which comprises target and/or subtractor ligand associated with separation means.

The term “antibody variant” shall be taken to refer to any synthetic antibodies, recombinant antibodies or antibody hybrids, such as, but not limited to, a single-chain antibody molecule produced by phage-display of immunoglobulin light and/or heavy chain variable and/or constant regions, or other immunointeractive molecule capable of binding to an antigen in an immunoassay format that is known to those skilled in the art.

The term “antibody derivative” refers to any modified antibody molecule that is capable of binding to an antigen in an immunoassay format that is known to those skilled in the art, such as a fragment of an antibody (e.g. Fab or Fv fragment), or an antibody molecule that is modified by the addition of one or more amino acids or other molecules to facilitate coupling the antibodies to another peptide or polypeptide, to a large carrier protein or to a solid support (e.g. the amino acids tyrosine, lysine, glutamic acid, aspartic acid, cysteine and derivatives thereof, NH₂-acetyl groups or COOH-terminal amido groups, amongst others). The term “antibody derivative” also refers to bispecific antibodies and cells expressing antibodies on their surface (such as chimeric antigen receptor T-cells).

By “density centrifugation” we include the meaning of the separation of items (e.g. cells, organelles, and macromolecules) according to their density differences. This separation is achieved by centrifugation using a density gradient of an appropriate solution, through which the items being separated move on the basis of their density.

The “Law of Mass Action” is a universal law of nature that is applicable under any circumstance. This law states that for the reaction:

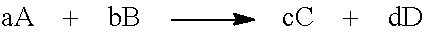

and if that system is at equilibrium at a given temperature, then the following ratio is a constant:

$$\frac{{{[ C ]}^{c} [ d ]}^{d}}{{{[ A ]}^{a} [ B ]}^{b}} = {K}_{eq}$$

where:
   A, B, C & D=are the participants in the reaction (reactants and products)
   a, b, c, & d=the coefficients necessary for a balanced chemical equation

And wherein the constant is calculated in terms of concentration (indicated by [ ]) and K has units M^{c+d-(a+b)}.

Examples embodying certain aspects of the invention shall now be described, with reference to the following figures.

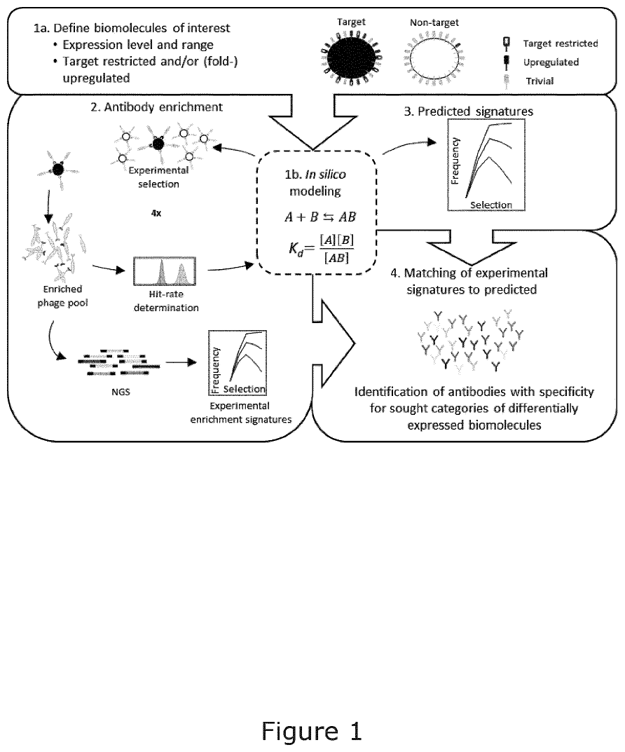

FIG. 1 shows a schematic outline of the method of the present invention First, target biomolecule categories of interest are defined by their absolute and relative expression levels in target versus non-target samples. In silico modeling of selection are used to optimise selection reaction parameters enabling enrichment of antibodies according to their affinity (KD)-driven binding to defined categories of differentially expressed target biomolecules and depletion of antibodies to trivial biomolecules expressed at similar levels in target and non-target samples. Second, the optimised reaction parameters are used to experimentally enrich antibodies in four consecutive selections. Resulting phage pools are analysed by massively parallel sequencing to provide antibody enrichment signatures, and a small subset of enriched antibodies is analysed to determine the fraction of target reactive binders (hit-rate) following each selection. Third, the hit-rates are incorporated in in silico modeling to generate predicted enrichment signatures for antibodies to defined categories of differentially expressed target biomolecules. Finally, experimentally obtained enrichment signatures of individual antibody clones are matched to in silico modelled enrichment signatures, in order to identify antibodies to sought categories of differentially expressed biomolecules

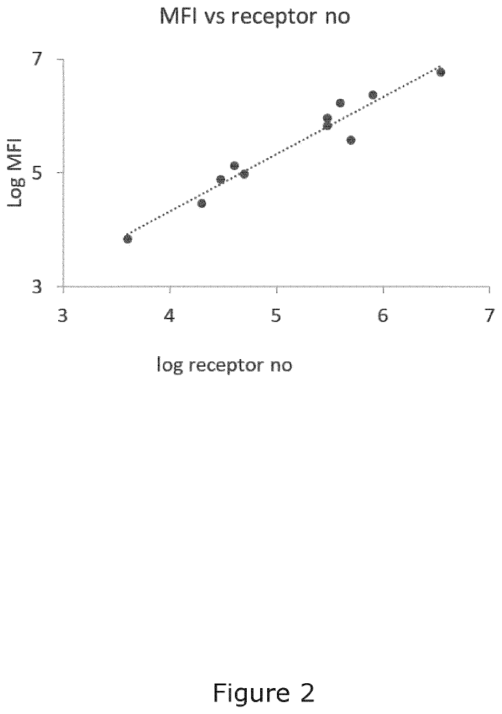

FIG. 2 shows antibody binding to target DU145 cells in flow cytometry. Calibration curve showing log MFI at saturated antibody concentration vs. log receptor number quantified using ABC beads.

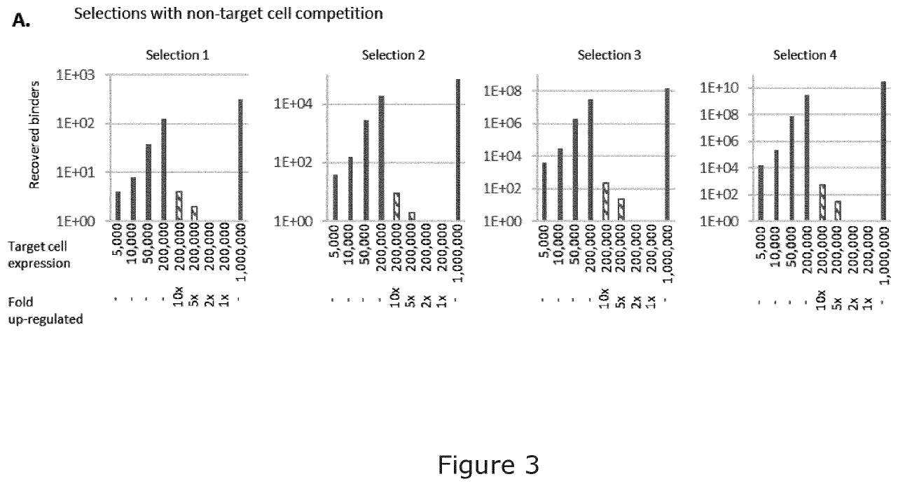

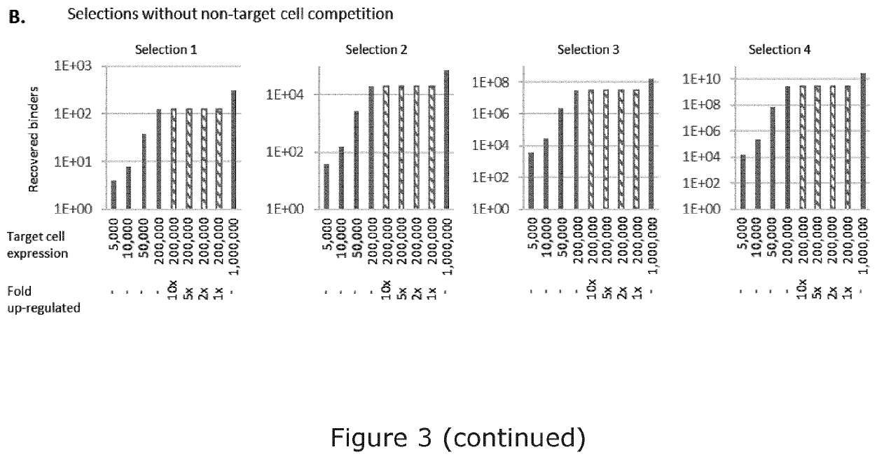

FIG. 3 shows in silico optimization of selection reaction parameters using the method of the present invention. Calculated recovery of 10 nM binders targeting receptors expressed at 5,000, 10,000, 50,000, 200,000 or 1,000,000 receptors/target cells with no expression on non-target cells, or receptors upregulated 10×, 5×, 2× or 1× on target cells compared to non-target cells using the optimised conditions. (A) Number of recovered binders in selections with non-target cell competition. (B) Number of recovered binders in selections without non-target cell competition.

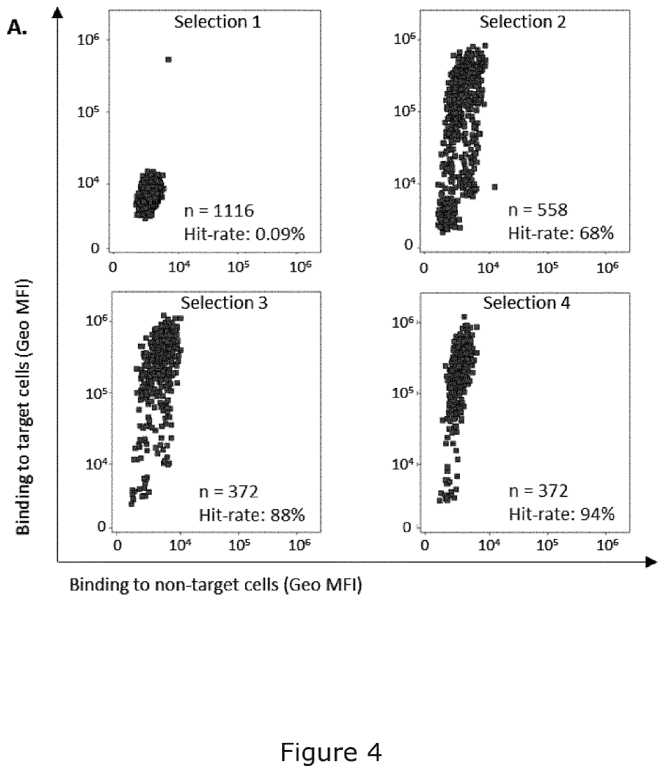

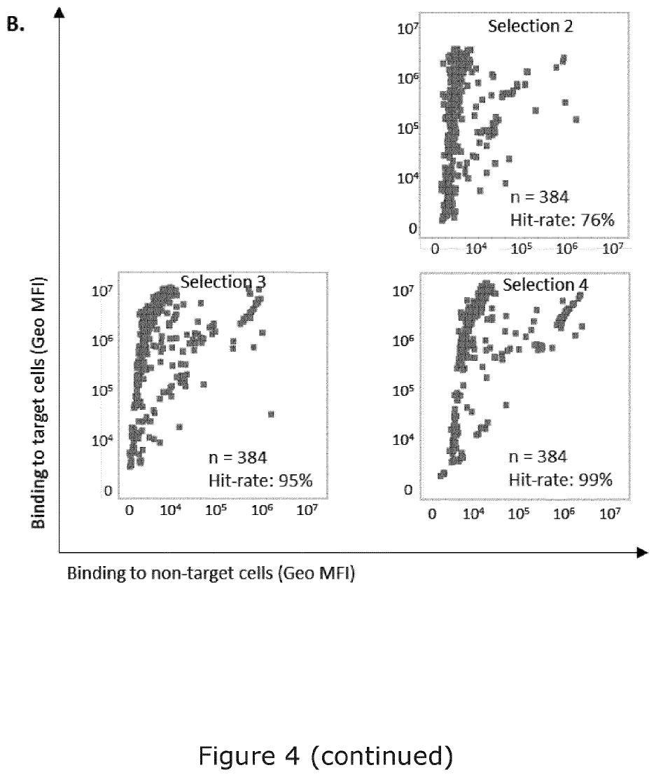

FIG. 4 shows hit-rate determination. Flow cytometry binding analysis showing the binding to target and non-target cells. (A) Analysis of clones from selection 1, 2, 3, and 4 with non-target cell competition. (B) Analysis of clones from selection 2, 3, and 4 without non-target cell competition. For selection 1 without competition, the hit-rate was estimated to 0.09%, the same as for selection 1 with competition.

FIG. 5 shows how predicted signatures model antibody enrichment according to targeted receptors absolute and relative expression levels. (A) Overview of ten reference receptors used to evaluate the accuracy of predicted signatures, showing the number of receptors on target and non-target cells. The selection conditions used in this study were optimised for discovery of antibodies targeting receptors in the grey area. (B) Predicted signatures and experimental outcomes. Black lines show in silico calculated enrichment signatures for antibodies targeting the reference receptors. Violin plots show enrichment of antibodies targeting these receptors in selections with non-target cell competition (upper panels of violin plots) and without non-target cell competition (lower panels of violin plots). The areas shown in the “Relevance” section indicate the range of diagnostically and therapeutically relevant receptors, and the therapeutic applicability of antibodies targeting these. The areas shown in the “Discovery strategy” section indicate whether antibodies targeting the receptors can be found with conventional screening technology and/or the method of the present invention.

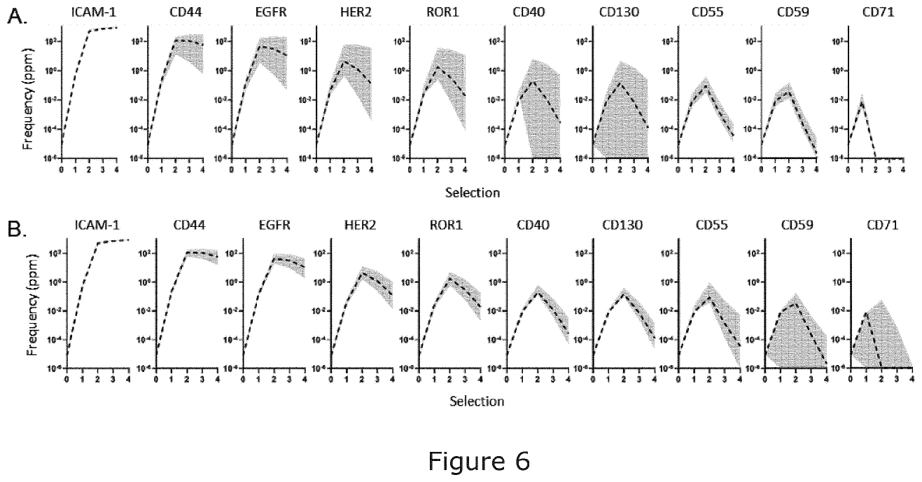

FIG. 6 shows in silico calculated enrichment signatures for ten reference receptors in selections with non-target cell competition. Shaded areas indicate the variation with different parameters. (A) Variation of antibody affinity. Dotted lines show the predicted enrichment signatures for 10 nM antibody affinity, grey areas indicate 10 times higher or lower affinity (1-100 nM). (B) Variation of number of cell surface receptors. Dotted lines show the predicted enrichment signatures for measured number of cell surface receptors according to Table 3, shaded areas indicate two times higher or lower receptor numbers.

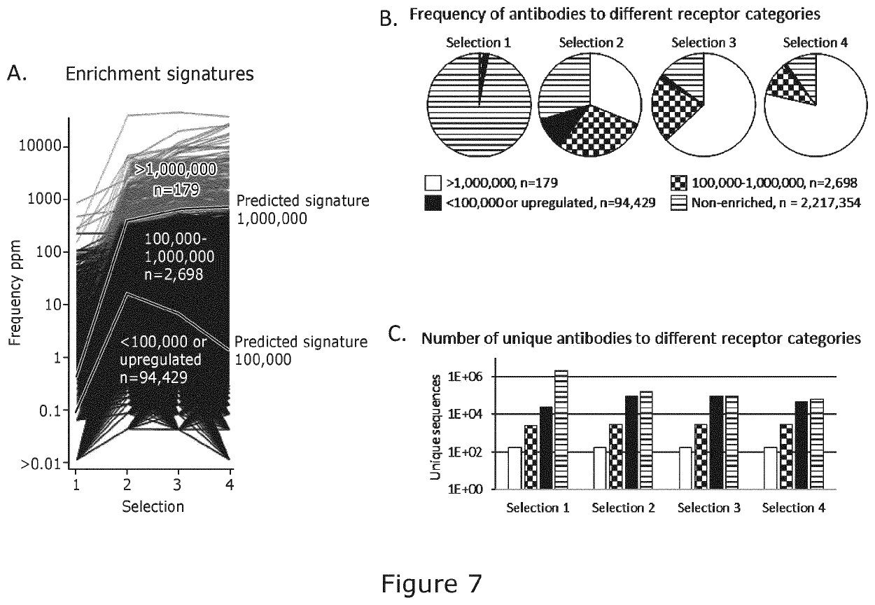

FIG. 7 shows antibody enrichment in four consecutive selections on target cells with non-target cell competition monitored by massively parallel sequencing. (A) Enrichment signatures showing antibody sequence frequencies after selection 1 to 4. Black lines show in silico predicted signatures for antibodies targeting receptors expressed at 1,000,000 and 100,000 copies/target cell with no expression on non-target cells. The antibodies were classified into three groups targeting receptors expressed at >1,000,000 copies/cell, 100,000-1,000,000 copies/cell or <100,000 copies/cell or upregulated receptors. n shows the total number of unique sequences within each group. (B) The frequency of antibodies to different receptor categories after selection 1-4. Antibodies classified as non-enriched are predicted to not bind target cells or to bind receptors expressed at similar levels on target and non-target cells. (C) The number of unique antibodies to different receptor categories after each selection.

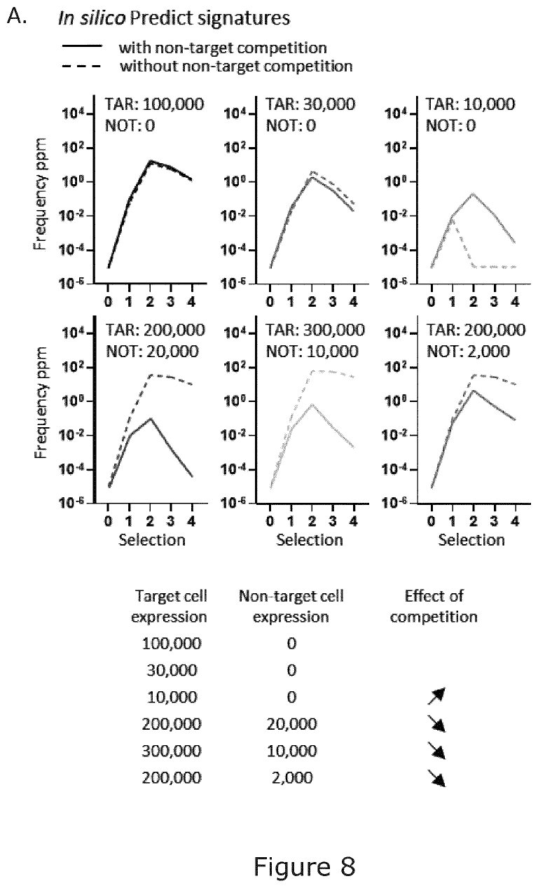

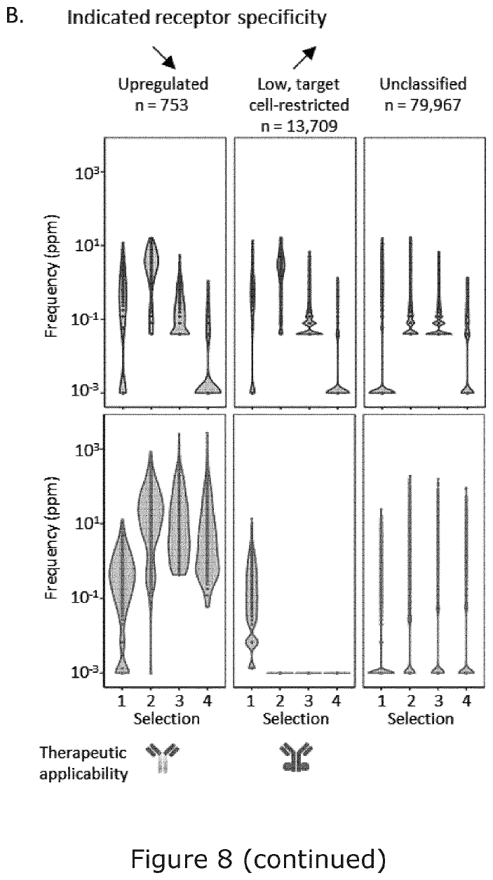

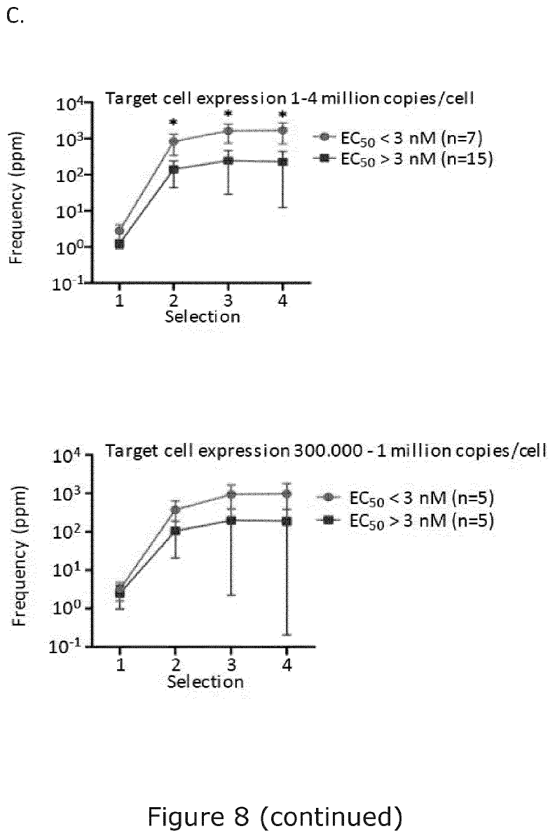

FIG. 8 shows enrichment signatures in selections with and without non-target cell competition. (A) The low frequency region in selections with non-target cell competition (lower group in FIG. 7A), represents antibodies targeting receptors expressed at <100,000 copies/target cell, but also receptors that are upregulated on target compared to non-target cells, as shown by in silico derived enrichment signatures (solid lines). In silico signatures from selections without non-target cell competition (dotted lines), indicate that these antibodies can be further classified guided by comparative analyses of enrichment signatures from selections with and without non-target cell competition. Lower panel show the direction of change in selections with non-target cell competition compared to selections without non-target cell competition for binders to receptors with indicated expression levels. (B) Antibodies classified as targeting receptors expressed at <100,000 copies/target cell (lower group in FIG. 7A), has been further divided based on signatures from selections without non-target cell competition. Violin plots in the upper panels show selections with competition, violin plots in the lower panels show selections without competition. Antibodies have been classified either as binders to upregulated receptors (decreasing frequency in selections with competition), binders to low expressed, target cell-restricted receptors (increasing frequency in selections with competition) or have not been classified (not clearly affected by competition selection). (C) Antibodies against target cell restricted receptors with similar expression levels were divided into two groups based on their EC₅₀ values in flow cytometry. The antibody frequency in the selected phage pools after selections 1˜4 is plotted as the mean frequency (ppm)±SEM. The upper panel shows the frequency of antibodies targeting receptors with an expression level of 1-4 million copies/cell and the lower panel shows the frequency of antibodies targeting receptors with an expression level of 300,000-1 million copies/cell. Antibodies with an EC₅₀ value<3 nM (circle data points) are more frequent than antibodies with an EC 50 value>3 nM (square data points) after selections 2, 3, and 4, but not after selection 1. The difference is significant for antibodies targeting receptors expressed at 1-4 million copies/cell (n=22). * indicates p<0 05 by Mann Whitney test (GraphPad Prism-Selection 2: p=0 046522; Selection 3: p=0 026175; Selection 4: p=0 033252).

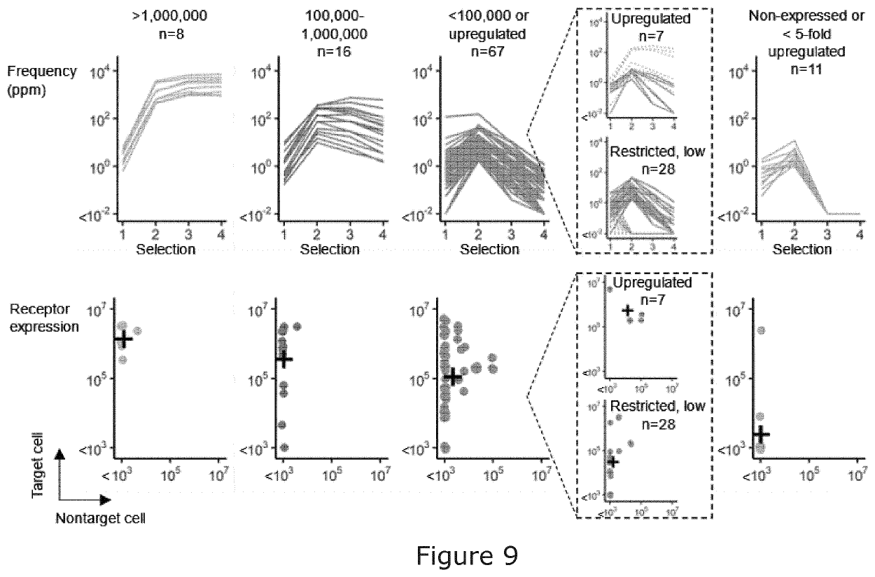

FIG. 9. A subset of antibodies from each enrichment signature group (top panel) was produced and tested for binding to target and non-target cells (bottom panel). The crosses in the lower panel represent the geometric mean of receptor expression on target and non-target cells. Inset: Antibodies in the <100,000 or upregulated group further classified as binding upregulated or restricted low expressed receptors based on comparative analyses of selections with non-target cell competition (solid lines) and selections without non-target cell competition (dotted lines).

### EXAMPLES

### Example 1—Deriving the Equations Used in the Experiments of the Examples

According to the Law of Mass Action, the interaction between an anti-ligand (A), its target receptor (B), and their complex (AB) is given by the equilibrium interaction:

$$A + B ⇌ AB$$

with the equilibrium dissociation constant or affinity

$${K}_{d} = \frac{[ A ] [ B ]}{[ AB ]}$$

The equilibrium interaction between (A) and (B) may be described as

$$Bound A ( bA ) ⇌ free A ( fA ) + free B ( fB ) \\ with \\ Kd = [ fA ] \times \frac{[ fB ]}{[ bA ]} \qquad ( 1 )$$

The total A or B ([A] or [B]) is the sum of free and bound A or B, i.e.,

$$[ A ] = [ fA ] + [ bA ] , and [ B ] = [ fB ] + [ bA ]$$

Therefore in (1) replacing [fA] by [A]-[bA], and [fB] by [B]-[bA], gives

$${K}_{d} = ( [ A ] - [ bA ] ) \times \frac{[ B ] - [ bA ]}{[ bA ]} \qquad ( 2 )$$

Which is rearranged to form

$$( {K}_{d} [ bA ] ) = ( [ A ] [ B ] - [ A ] [ bA ] ) - ( [ B ] [ bA ] - {[ bA ]}^{2} ) \\ 0 = {[ bA ]}^{2} - ( [ A ] + [ B ] + {K}_{d} ) [ bA ] + [ A ] [ B ]$$

This equation has the solution

$$[ bA ] = \frac{( [ A ] + [ B ] + {K}_{d} )}{2} \pm \sqrt{\frac{{( [ A ] + [ B ] + {K}_{d} )}^{2}}{4} - [ A ] [ B ]}$$

where the negative root is the relevant one

$$[ bA ] = \frac{( [ A ] + [ B ] + {K}_{d} )}{2} - \sqrt{\frac{{( [ A ] + [ B ] + {K}_{d} )}^{2}}{4} - [ A ] [ B ]}$$

Substituting concentrations for number of antibodies/the number of particles per mole (N_{A})/volume (V) yields

$$\frac{bA}{( {N}_{A} V )} = \frac{( \frac{A}{( {N}_{A} V )} + \frac{B}{( {N}_{A} V )} + {K}_{d} )}{2} - \sqrt{\frac{{( \frac{A}{( {N}_{A} V )} + \frac{B}{( {N}_{A} V )} + {K}_{d} )}^{2}}{4} - \frac{AB}{{( {N}_{A} V )}^{2}}}$$

or simplified

$$bA = \frac{A + B + {K}_{d} {N}_{A} V}{2} - \sqrt{\frac{{( A + B + {K}_{d} {N}_{A} V )}^{2}}{4} - AB} \qquad ( 3 )$$

where
   bA=number of receptor bound anti-ligands
   A=total number of anti-ligands A
   B=total number of target receptors B
   K_{d}=Antibody affinity (M)
   V=the reaction volume (dm³)
   N_{A}=Avogadro's constant (6.022×10²³ particles mole⁻¹)

If target and non-target cells are mixed the total number of receptors (B) will be:

$$B = ( {B}_{T} {C}_{T} + {B}_{N} {C}_{N} )$$

where
   C_{T}=the number of target cells
   C_{N}=the number of non-target cells
   B_{T}=the number of receptors on C_{T}
   B_{N}=the number of receptors on C_{N}

The number of anti-ligands A bound to receptors B on target cells at equilibrium will be equal to the total number of bound anti-ligands on target and non-target cells multiplied by the ratio between receptors on target cells and the total number of receptors (receptors on both target and non-target cells):

$${bA}_{T} = bA \times \frac{{B}_{T} {C}_{T}}{{B}_{T} {C}_{T} + {B}_{N} {C}_{N}} \qquad ( 4 )$$

Furthermore, the combination of equations (3) and (4) yields

$${bA}_{T} = ( \frac{A + B + {K}_{d} {N}_{A} V}{2} - \sqrt{\frac{{( A + B + {K}_{d} {N}_{A} V )}^{2}}{4} - AB} ) \times \frac{{B}_{T} {C}_{T}}{{B}_{T} {C}_{T} + {B}_{N} {C}_{N}} \qquad ( 5 )$$

Since not all antibodies are recovered after a selection, the number of recovered antibodies (rA_{T}) is given by:

$${rA}_{T} = ( ( \frac{A + B + {K}_{d} {N}_{A} V}{2} - \sqrt{\frac{{( A + B + {K}_{d} {N}_{A} V )}^{2}}{4} - AB} ) \times ( \frac{{B}_{T} {C}_{T}}{{B}_{T} {C}_{T} + {B}_{N} {C}_{N}} ) ) \times E \times Y \qquad ( 6 )$$

where
   E=the fraction of anti-ligands eluted from target cells
   Y=the fraction of recovered target cells

In the n-CoDeR® library, the average copy number of each anti-ligand is 2,000 with a 10% display level. Hence, A in selection 1 is set to 200. In succeeding selections, A is calculated as the number of recovered anti-ligands (rA_{T}) in previous selection multiplied by the amplification factor. The amplification factor is experimentally determined (or set to 10,000, 100,000 and 10,000 between selection 1-2, 2-3 and 3-4 respectively during selection optimisation). E and Y are experimentally determined (or set to 0.5 during selection optimisation).

The frequency of a recovered anti-ligand in the selected phage pool (FA_{T}) is calculated as:

$${FA}_{T} = \frac{{rA}_{T}}{( {\sum }_{1}^{n} {rA}_{T} )} \times HR \qquad ( 7 )$$

where
   FA_{T}=frequency of recovered anti-ligand
   rA_{T}=number of recovered anti-ligands

$${\sum }_{1}^{n} {rA}_{T} = \text{Total number of recovered anti-ligands}$$

HR=hit-rate, fraction of anti-ligands specific for the target cells (experimentally determined)

$${\sum }_{1}^{n} {rA}_{T} equals the total number of anti - ligands binding to receptors$$

$$that are considered relevant .$$

### Example 2—Overview of the Study with Materials and Methods

### Overview of the Study

The results of this study are presented in Examples 3 to 6 below. This study uses novel prediction-based methodology combining in silico modeling of antibody-enrichment with iterative experimental antibody-display selection and massively parallel sequencing analyses, to rationally identify tens-of-thousands of sequences encoding antibodies to a priori unknown, differentially expressed biomolecules, which are present at unknown levels in complex biomolecule samples. When applied to a naïve phage-display human scFv antibody library to select antibodies to surface receptors differentially expressed on cancer compared with immune cells, 105 enriched antibody sequences were predicted to bind differentially expressed biomolecules. Following subset sampling, and expression in full-length human IgG format, 90% of these antibodies were verified to bind cancer cell-associated receptors, which were expressed across therapeutically and diagnostically relevant ranges (from a few thousand to millions of receptors per cell). The method of the present invention is broadly applicable to large molecular libraries coupling genotype to phenotype during screening of complex biomolecule populations, e.g. body fluids, immune or cancerous cells, or pandemic microorganisms.

The present invention integrates computational modelling with experimental (phage-, yeast- or ribosome display) antibody selection, and massively parallel sequencing, to rationally enrich and identify antibodies to a priori unknown biomolecules that are differentially expressed between two samples, from large antibody libraries coupling genotype to phenotype (FIG. 1).

First, categories of differentially expressed target biomolecule specificities to model are defined by hypothetical biomolecules' absolute and relative expression levels, ranging from lowest to, by approximate 10-fold serial increments, highest estimated in target and non-target samples. Selection reaction parameters enabling enrichment of antibodies according to their affinity (K_{D})-driven binding to biomolecules of interest and, in the case of competition selection, depletion of trivial antibodies specific to biomolecules expressed at similar levels in target and non-target samples, are identified by in silico modeling of selection (FIG. 3).

Second, experimental selection of the antibody library, and parallel sequence analyses of retrieved antibody pools, is performed to provide: (1) experimentally enriched antibody sequences, (2) their associated enrichment signatures, and (3) the fraction of displayed antibodies that has been enriched in an antibody and biomolecule-dependent manner (the hit-rate).

Third, hit rates are incorporated in in silico modeling to generate predicted/reference enrichment signatures for antibodies to sought categories of differentially expressed target biomolecules. Predicted enrichment signatures may be experimentally validated using reference binder sequences to molecules with known expression in target and non-target antigen populations (optional).

Finally, experimentally obtained enrichment signatures of individual antibody clones are matched to predicted enrichment signatures, so as to identify antibody sequences (genotypes) encoding specificity (phenotypes) for sought categories of differentially expressed target biomolecules (FIG. 1).

### Materials and Methods for Examples 3 to 6

### Cells

Cell lines DU145 (prostate carcinoma) and Jurkat (clone E6-1, acute T cell leukaemia), were obtained from ATCC and cultured according to the supplier's instructions. DU145 cells were stimulated with INF-γ (R&D Systems), 27 ng/ml for 16-24 h before use.

### In Silico Optimisation of Selection Conditions

The number of target and non-target cells needed to recover and enrich 10 nM antibodies against (1) receptors with ≥5,000 copies or more per target cell and no expression on non-target cells and (2) receptors upregulated at least 5 times and with ≥200,000 copies on target cells compared to non-target cells was calculated using Equation 6 described in Example 1. The total number of antibodies A in selection 1 was set to 200, since the average copy number of each antibody in the n-CoDeR® library is 2,000 with a 10% display level. In succeeding selections, A was calculated as the number of recovered antibodies (rA_{T}) in previous selection multiplied by the amplification factor. Amplification factors of 10,000, 100,000 and 10,000 between selection 1-2, 2-3 and 3-4, respectively, were used in calculations and indicated to promote enrichment of sought specificities, and depletion of trivial specificities. The fraction of antibodies eluted from target cells E and the fraction of recovered target cells Y were set to 0.5.

### Predict-Guided Cell Selections

For selections with non-target cell competition, DU145 target cells were harvested, washed, biotinylated with EZ-Link™ Sulfo-NHS—SS-Biotin (Thermo Fisher Scientific) and labelled with anti-biotin microbeads (Miltenyi Biotec) according to the manufacturer's instructions. Labelled target cells (10, 2.5, 5 or 5 million cells in selection 1, 2, 3 and 4 respectively) were mixed with approximately 1,000 times excess of Jurkat non-target cells and incubated with the n-CoDeR® scFv phage display libraryl (BioInvent International) at +4° C., overnight, on a rocking platform. The cell-phage mixture was loaded on a MACS column followed by extensive washing. After elution of target cells from the column, phages bound to target cells were recovered and amplified for use in consecutive selections. Phagemid DNA was purified and genes encoding scFv were used for production of soluble scFv, as described previously (Ljungars et al, 2019), and Illumina sequenced.

In selections without non-target cell competition, phages were incubated with 10, 2.5, 5, or 5 million DU145 cells in selection 1, 2, 3 and 4 respectively for 4 h at +4° C., on a rocking platform. Cells were washed with Phosphate Buffered Saline (PBS) four times before phages were recovered and proceeded as described above.

### Hit-Rate Determination

300-1,100 ScFv containing supernatants from each selection were filtered (0.45 μm Millipore) and 25 μL/well was added to a 1:1 mixture of DU145 cells and CellTrace™ CSFE-labelled (Thermo Fisher Scientific) Jurkat cells (50,000 cells in 25 μL PBS+0.5% BSA/well) and left to bind for 1 h at +4° C. After washing, scFv binding to live cells (eBioscience™ Fixable Viability Dye eFluor™ 780, Thermo Fisher Scientific) was detected using anti-His-AF647 (R&D Systems) and analysed by flow cytometry (iQue, Intellicyt Sartorius FortCyt v 8.0). The hit-rate was determined as the fraction of analysed clones with Mean Fluorescence Intensity on target cells at least three times higher than an isotype control.

### In Silico Calculation of Predicted Enrichment Signatures

In silico predicted enrichment signatures, defined as the expected antibody frequencies observed over four consecutive selection rounds of 10 nM antibodies targeting receptors with varying expression profiles were calculated using Equations 6 and 7 described in Example 1. The total number of antibodies A in selection 1 was set to 200, since the average copy number of each antibody in the n-CoDeR® library is 2,000 with a 10% display level. In successive selections, A was calculated as the number of recovered antibodies (rA_{T}) in previous selection multiplied by the amplification factor (experimentally determined). The fraction of antibodies eluted from target cells E and the fraction of recovered target cells Y were experimentally determined. Phage-antibody binding to biomolecules expressed throughout the experimentally determined expression range (5×103 to 4×106 copies/target cell) were modelled, assuming same median affinities (KD=10 nM), and the same number of antibodies specific for different categories of biomolecules, being present in the unselected naïve antibody library.

### Sequence Library Preparation and Illumina Sequencing

Phagemide DNA was purified from enriched phage pools using the QIAprep Spin Miniprep Kit (Qiagen). One-step PCR using PfuUltra II Fusion HS DNA Polymerase (Agilent) was performed to amplify scFv encoding genes from phagemid DNA and attach Illumina adaptors and indexes to the samples. Reaction volume was 50 μl/sample, with 50 ng template and 0.2 μM of each primer. Samples for MiSeq sequencing were amplified using two different primer pairs; the first pair covers CDR-H1, CDR-H2 and CDR-H3 and the second pair covers CDR-L1, CDR-L2, CDR-L3 and CDR-H3. Samples for NextSeq sequencing were amplified using a primer pair that covers CDR-H3. Reverse primers include a 10-bp index sequence to facilitate multiplexing. The primer sequences used are provided in Table 2. PCR amplification was carried out with the following conditions: 95° C./2 min; 12 cycles of 95° C./20 s, 62° C./30 s, 72° C./30 s; followed by 72° C./3 min. PCR products were purified from a 2% agarose gel (MinElute Gel Extraction Kit, Qiagen), quantified (Qubit™ dsDNA HS Assay Kit, Thermo Fisher Scientific) and analysed for purity and size on Agilent 2100 Bioanalyzer using the Agilent 1000 DNA kit and Agilent 2100 Expert software (version B.02.08.SI648 (SRI)). The concentration of pooled sequence libraries was measured using the KAPA Library Quant Kit Universal qPCR Mix (Roche, KK4824).

TABLE 2
Primer sequences

| Primer | Sequence (5′-3′ directionality) |
|---|---|
| Forward | AATGATACGGCGACCACCGAGATCTACACTCTTTCCCTACACGACGCTCTTCCGATCT |
| MiSeq1 | ttccctgagactctcctgtgcagcctctggattcacctt |
| Forward | AATGATACGGCGACCACCGAGATCTACACTCTTTCCCTACACGACGCTCTTCCGATCT |
| MiSeq2, | tagagccgaggacactgccgtgtattactgt |
| NextSeq | |
| Reverse | CAAGCAGAAGACGGCATACGAGAT-[10 ntINDEX]-GTGACTGGAGTTCAGACGTGT |
| MiSeq1, | GCTCTTCCGATCTcgctgctcacggtgaccagtgtaccttggcccca |
| NextSeq | |
| Reverse | CAAGCAGAAGACGGCATACGAGAT-[10 ntINDEX]-GTGACTGGAGTTCAGACGTG |
| MiSeq2 | TGCTCTTCCGATCTgtcagcttggttcctccgccgaa |

Upper case = sequencing adaptors; Lower case = scFv primers.

Samples for MiSeq were combined on a flow cell (Illumina MiSeq Reagent Kit v3 (600-cycles)) with 10% PhiX added and sequenced on MiSeq using paired-end reads to a median depth of 8.5 million usable reads/sample. Samples for NextSeq were combined on four flow cells (Illumina NextSeq 500/550 High Output Kit v2.5 (300 cycles)) with 25% PhiX added and sequenced on Illumina NextSeq 500 using single reads to a median depth of 25 million usable reads/sample. Base-calling and demultiplexing were performed using bcl2fastq version 2.20.0.422, quality of resulting fastq files were then inspected using fastQC version 0.11.8. with default settings. For each sequence, the number of reads was normalised to the total number of reads in corresponding pooled library. Sequences with less than two reads were omitted from further analysis.

### Identification of Reference Receptors Covering the Expression Range of Diagnostically and Therapeutically Relevant Targets

Ten cell surface receptors with varying gene expression profiles on target cells (DU145) and non-target cells (Jurkat) were identified through searches in the Cancer Cell Line Encyclopedia (Broad Institute, Cancer cell line encyclopedia, 2019) and literature studies (Liu et al, 2000) (see Table 3). Cell surface expression of these was measured by flow cytometry, using labelled antibodies (see Table 4) and quantification beads (Bang Laboratories, 815B) according to the manufacturer's instructions.

TABLE 3
Number of receptors expressed on target and non-target
cells, experimentally determined by flow cytometry.

| | Target cell expression | Non-target cell expression |
|---|---|---|
| Receptor | (receptors per cell) | (receptors per cell) |
| ICAM-1 | 4,700,000 | Below detection limit |
| CD44 | 400,000 | Below detection limit |
| EGFR | 200,000 | Below detection limit |
| HER2 | 50,000 | Below detection limit |
| ROR1 | 30,000 | Below detection limit |
| CD40 | 10,000 | Below detection limit |
| CD130 | 8,000 | Below detection limit |
| CD55 | 200,000 | 20,000 |
| CD71 | 200,000 | 100,000 |
| CD59 | 400,000 | 100,000 |

TABLE 4
Labelled antibodies/recombinant reference receptors

| Receptor | Recombinant protein | Commercial antibody |
|---|---|---|
| ICAM-1 | R&D Systems # 720-IC | BD Biosciences # 559771 |
| CD44 | Sino Biological # 12211-H08H | BD Biosciences # 559942 |
| EGFR | Sino Biological # 10001-H08H | BD Biosciences # 555997 |
| HER2 | Sino Biological # 10004-H08H | BD Biosciences # 340552 |
| ROR1 | Sino Biological # 13968-H08H | BD Biosciences # 564474 |
| CD40 | Produced in-house | BD Biosciences # 555589 |
| CD130 | Sino Biological # 10974-HCCH | BD Biosciences # 555757 |
| CD55 | Sino Biological # 10101-H08H | BD Biosciences # 561901 |
| CD59 | Sino Biological # 12474-H08H | Miltenyi # 130-120-048 |
| CD71 | Sino Biological # 11020-H07H | BD Biosciences # 551374 |

### Generation of Antibodies Targeting Reference Receptors

The n-CoDeRR library or amplified phages from cell selection 2 with non-target cell competition was used for selection against recombinant reference receptors (Table 4) using Polystyrene beads (Polysciences, 17175) coated with 25 pmole/bead, 4 beads/receptor as described previously (Ljungars et al, 2019). For selections starting with the n-CoDeR® library, a second selection on recombinant protein was performed followed by a third selection on DU145 cells. Phage-bound antibodies were converted to soluble scFvs, expressed, and analysed by flow cytometry as described above for Hit-rate determination. ScFvs binding to DU145 cells were analysed for binding to respective receptor in ELISA. Reference receptors (Table 4) were coated to plates overnight at +4° C. The next day scFv supernatant diluted 1:4 in PBS with 0.05% Tween 20 and 0.45% fish gelatin (both from Sigma-Aldrich) were left to bind the washed ELISA plate for 1 h at room temperature. Bound scFv was detected using an AP-conjugated anti-FLAG M2 antibody (Sigma-Aldrich) followed by addition of a luminescent substrate (CDP Star Emerald II, Thermo Fisher Scientific) and plates were read in a plate reader (Tecan Ultra with Tecan Magellan v.3.0). All receptor binding clones were cherry-picked, grown overnight in 96-well microtiter plates and Sanger sequenced.

### Prediction-Guided Antibody Classification

Antibody frequency throughout the consecutive selections (F_{S1}, F_{S2}, F_{S3}, F_{S4},) were obtained from the NextSeq data. The antibodies were classified in three steps.

First, signatures from selections with non-target cell competition were used to classify the antibodies as binders if F_{S2}>F_{S1} while F_{S2} and F_{S3}>0. Antibodies not meeting these criteria were classified as non-enriched.

Second, antibodies in the binder group were classified into three binder types, again based on selections with non-target cell competition. F_{S4} was compared to predicted signatures for binders with 10 nM affinities for receptors expressed at 100,000 and 1,000,000 copies/target cell with no non-target cell expression (1.3 ppm and 750 ppm respectively) to guide the classification.
1)>1,000,000 (Antibodies with a frequency signature higher than the 1,000,000 predicted signature)-inclusion criteria: F_{S4}>750 ppm
2) 100,000-1,000,000 (Antibodies with a frequency signature between the 100,000-1,000,000 predicted signatures)-inclusion criteria: 1.32 ppm<F_{S4}<750 ppm
3)<100,000 (Antibodies with a frequency signature lower than the 100,000 predicted signature)-inclusion criteria: F_{S4}<1.32 ppm

Third, antibodies classified as <100,000 were further classified by comparing signatures from selections with and without non-target cell competition.

$$1 ) Upregulated - inclusion criteria \frac{{F}_{S 4} without competition}{{F}_{S 4} with competition} > 100 \\ or , \\ if {F}_{S 4} with competition = 0 ; \\ {F}_{S 4} without competition > 0 \\ and \\ \frac{{F}_{S 3} without competition}{{F}_{S 3} with competition} > 10$$

2) Low expressed, target cell-restricted-inclusion criteria F_{S1} #0 and F_{S2}, F_{S3}, F_{S4}=0 (all without non-target cell competition)
3) Unclassified—not meeting any of the criteria above.

### Production of IgG

Full scFv sequences needed for clone synthesis was obtained from the Miseq data. In brief, VH and VL+CDR-H3 sequences were combined based on the CDR-H3 sequence. In cases where one CDR-H3 was associated with more than one set of VH and/or VL sequences, the frequencies in the two libraries were used to join the correct VH/VL pair. Antibody genes were synthesised (Twist Bioscience), ligated into a vector containing genes encoding the heavy and light chain constant regions of a human IgG1 antibody, produced in HEK293 cells, and purified as described previously (Ljungars et al, 2018).

### Confirmatory Binding Analysis of Prediction-Guided Antibodies

Purified IgG were diluted to 100 μg/mL and titrated in 25 μL PBS+0.5% BSA and added to 50,000 DU145 (target) and Jurkat (non-target) cells/well and left to bind for 1 h at +4° C. After washing, IgG bound to live cells was detected using an APC-conjugated anti-human-Fc antibody (Jackson Immunoresearch, 109-136-098) together with a live/dead cell marker (SYTOX green, Thermo Fisher Scientific) and analysed by flow cytometry (iQue, Intellicyt Sartorius using FortCyt v 8.0). In order to generate a calibration curve to transform MFI at saturated binding to receptor number, a subset of IgGs with different signal intensity at saturated cell binding concentration was selected for receptor number determination. Purified IgGs were labelled with AF647 using an Alexa Fluor 647 carboxylic acid succinimidyl ester (ThermoFisher Scientific) according to the manufacturer's instructions. Labelled antibodies were used for receptor number determination using calibration beads (Bang Laboratories, 816) according to manufacturer's instructions (FIG. 2). The assay detection limit (receptor number for isotype control) was 1,000 receptors/cell.

### Quantification and Statistical Analysis

Statistical analysis was performed using GraphPad Prism 9 and conducted by a Mann-Whitney test with *P≤0.05 (FIG. 8C).

### Example 3—Predicted Enrichment Signatures Accurately Model Enrichment of Binders According to Targeted Receptors' Absolute and Relative Expression Levels

The method of the present invention was used to screen a large (>10¹⁰ members) naïve human antibody library (Soderlind et al, 2000) for antibodies to cell surface receptors differentially expressed between two cell types-DU145 prostate cancer (target) cells compared with Jurkat T (non-target) cells.

In a first step, categories of differentially expressed surface receptors covering a wide dynamic expression range, spanning a few thousand (1,000s) to millions (1,000,000s) of receptors per cell, were defined. Any receptor upregulated five-fold or more on target compared with non-target cells was deemed to potentially be of therapeutic or diagnostic interest. Thus, selection reaction parameters enabling enrichment of relevant antibody binders to >five-fold upregulated receptors and removal or deselection against trivial binders to <five-fold upregulated receptors on target versus non-target cells, were sought. In silico modelling identified a selection protocol that should result in enrichment of high affinity (K_{D}≤10 nM) antibodies to receptors expressed throughout this range (103 to 106 receptors/cell), and which were at least 5-fold upregulated on target relative to non-target cells (FIG. 3A). The resulting protocol included application of positive and negative selection pressures in the form of target cells and excess of non-target cells, respectively. Modelling indicated that, in the absence of negative selection pressure, selection would fail to reduce trivial antibodies specific to receptors equally expressed, or <5-fold upregulated on target compared with non-target cells, and would skew enrichment towards highly expressed, rather than differentially expressed, receptors (FIG. 3B).

Four rounds of experimental selections were performed according to the above in silico optimised protocols (i.e. selections with or without competition). It was thought that enrichment signatures based on four (compared with fewer) data points, generated using selection with or without competition, would help discriminate antibodies to varying types of differentially expressed receptors, e.g. those with expression restricted to target cells but varying by expression level (restricted receptors), and those upregulated on target cells, i.e. also expressed on non-target cells (upregulated receptors). The fraction of phage antibodies that had been enriched in an antibody and target cell receptor-dependent manner in each selection round (i.e. the hit-rate) was determined by flow-cytometry following conversion of phage antibodies to scFv antibody format (FIG. 4).

Using the experimentally determined hit-rates, and a fixed K_{D} value equal to 10 nM, cell receptor-specific phage antibody enrichment was modelled in silico according to antibodies' K_{D}-driven equilibrium binding to categorised receptors. To experimentally validate predicted enrichment signatures, candidate reference cell surface receptors, with expression profiles matching categorised receptors, were identified by probing the Broad Institute Cancer Cell Line Encyclopedia (CCLE) for differentially expressed cell surface receptor-encoding genes in target compared with non-target cells. Flow-cytometry based analyses, using commercially available antibodies and ABC-quantification beads, verified that ICAM-1, CD44, EGFR, HER2, ROR1, CD40, CD130, and CD55, were differentially expressed on targets compared with non-targets, while CD59 and CD71 represented receptors<5-fold upregulated on target cells relative to non-target cells (FIG. 5A, Table 3). The numbers of molecules per target cell of restricted receptors spanned 103 (CD130), 104 (CD40, ROR1, HER2), 105 (CD44, EGFR), up to 106 receptors (ICAM-1) per target DU145 cell, with no detectable expression on non-target Jurkat cells. Upregulated receptors CD55, CD59 and CD71 were similarly expressed on target cells (2-4×105 receptors/cell) but were >5-fold (CD55, 10-fold) and <5-fold (CD59, 4-fold and CD71, 2-fold) upregulated on target compared with non-target cells (FIG. 5A, Table 3). Antibodies, and their coding sequences, (n=4 to 69) to each of the reference receptors were identified by selections using recombinant extracellular domains of each of each of the reference receptors (see Example 2), enabling assessment of enrichment profiles of antibodies to biomolecules with established expression differences between target and non-target cells.

Experimental enrichment of antibodies specific to reference receptors was monitored by massively parallel sequencing of the antibody pools obtained by experimental cell-based selections, and was compared to that predicted by in silico modeling (FIG. 5B). A strong correlation between in silico-predicted and experimentally determined median frequencies of antibodies to the differently expressed reference receptors was observed when the antibody-receptor equilibrium was modeled according to K_{D}=10 nM (FIG. 5B). This value corresponds well to median affinities of antibodies to diverse cell surface receptors of different types isolated from herein used n-CoDeR® antibody library (Roghanian et al, 2015; Fransson et al., 2006; Schiopu et al, 2007). Similar strong fits between in silico predicted and experimentally determined frequencies were observed for selection reactions with or without non-target cell competition (FIG. 5B). Notably, the signatures for antibodies to low expressed, target cell restricted receptors (CD40 and CD130), and upregulated receptors (CD55), were very similar in selections with non-target cell competition but differed widely in selections without competition.

Since selection of antibodies from highly diversified libraries will generate receptor-specific antibodies of varying affinity, antibody enrichment was additionally modelled according to 10-fold higher (1 nM) or 10-fold lower (100 nM) affinities. While enrichment signatures were overall similar, the modelled antibody frequencies increased with higher affinity and decreased with lower affinity (FIG. 6A). Enrichment signatures were more sensitive to antibody affinity the lower the targeted receptor was expressed. This correlated with the larger variation in frequencies observed for antibodies to low expressed (CD130, CD40, ROR1, HER2) compared with highly expressed (ICAM-1, CD44, EGFR) receptors (FIG. 5B). To assess how possible errors in receptor quantification might affect antibody enrichment signatures, enrichment according to 2-fold higher or 2-fold lower numbers than experimentally determined was modelled. This mainly affected signatures of antibodies to upregulated receptors (CD71, CD59, CD55; FIG. 6B).

In summary, in silico modeled enrichment of target receptor-specific antibodies closely mirrored experimental antibody enrichment, which was driven by targeted receptors' absolute and relative expression levels on target and non-target cells (FIG. 5B).

### Example 4—Enrichment Profiles Indicate Tens-of-Thousands of Antibodies to Receptors Differentially Expressed at a Wide Dynamic Range

The fact that the method accurately modeled enrichment of reference antibodies to receptors expressed over a wide dynamic range, supported its potential utility as a discovery tool to identify antibodies to targets associated with distinct therapeutic and diagnostic potential (FIG. 5B). The value of such a discovery tool would largely be determined by the number of unique binders to differentially expressed targets that the technology generates. To assess this, the in silico-optimised and selected antibody pools were queried for antibody sequences with enrichment profiles matching in silico predicted signatures of antibodies specific for different categories of therapeutically or diagnostically relevant receptors (FIG. 5B). Binders developed for diagnostic purposes do not depend on function-modulating properties and could therefore be specific to any differentially expressed receptor, regardless of expression level. Similarly, antibodies that rely purely on blockade of ligand-receptor signaling (e.g. anti-IL-6R) (Sebba, 2008), could be specific to receptors expressed over a wide dynamic range. In contrast, Fc-dependent and empowered therapeutic antibodies mediating, for example, ADCC and CAR-T cell specificity, owing to their powerful cytolytic nature, require low (Fc-dependent) or no (empowered) receptor expression on critical normal cells and tissue. Accordingly, antibodies with enrichment signatures matching those predicted to represent specificity for restricted receptors expressed at (1)>10⁶ molecules/target cell, (2) 10⁵ to 10⁶ molecules/target cell, or (3)<10⁵ molecules/target cell or being >5-fold upregulated on target compared with non-target cells, were quantified by analysing frequencies of individual antibody sequences in phage-antibody pools eluted in each of the four rounds of experimental selection.

Analysis of the complete sequence data set indicated the presence of tens-of-thousands of antibodies to either receptor category (FIG. 7A). Signature-guided analyses indicated antibodies specific for the most highly expressed receptors to dominate binder pools (10² to 10⁵ ppm/sequence) following two or more rounds of panning, and conversely, antibodies specific for lower expressed target cell restricted receptors, or upregulated receptors, to be rare (10⁻² to 10 ppm/sequence) (FIG. 7A-7B). Interestingly, and in contrast to their low frequency, the number of antibody clones specific for upregulated or target-restricted receptors with low expression was indicated to exceed 10⁴ by the same analyses (FIG. 7C).

The data presented above supports the notion that the method of the present invention enables discovery of antibodies to receptors across medically relevant expression ranges, including low expressed, and upregulated receptors (FIG. 7). Importantly, the observations that antibodies to low expressed and upregulated receptors were present at very low frequency (one or less clones per million) in selected pools, are consistent with the observed shortcoming of existing methods to generate such antibodies. In the absence of integrated computational modeling, and informed enrichment signatures identified in data generated by massively parallel sequencing, robust identification of these specificities would require production, and labour-intensive cell-based screening, of millions of antibody clones.

### Example 5—Predictions Enable Preferential Discovery of Binders with Distinct Therapeutic and Diagnostic Potential

Detailed analyses of antibodies belonging to the least frequent, but by numbers greatest, group (FIG. 7A, lower group) showed that, in addition to antibodies to low expressed (10³ to 10⁵ receptors/cell) restricted receptors, e.g. HER2, ROR1 and CD130, it included clones specific for intermediate to high expressed (105 to 106 receptors/cell) upregulated receptors, e.g. CD55 (FIG. 5B). As discussed above, these two target expression profiles are associated with therapeutic potential with empowered and function-blocking antibodies, respectively. Depending on the intended therapeutic modality, it would therefore make sense to focus on either type of antibody specificity.

The observations in Example 4 above demonstrated that enrichment signatures for antibodies to low expressed, restricted receptors and upregulated receptors were similar in selections with non-target cell competition but differed markedly in selections without competition (FIG. 5B). This suggested that comparative analyses of enrichment signatures, generated from selections with and without competition, could potentially be used to discriminate sequences encoding antibodies with therapeutic potential as empowered (to restricted receptors) or function-blocking (to upregulated receptors) antibodies. Supporting this notion, in silico modelled enrichment signatures indicated decreasing frequencies of antibodies to upregulated receptors and, depending on expression level, increasing or unaltered frequencies of antibodies to restricted receptors, in selections with, compared to without, non-target cell competition (FIG. 8A). These observations are consistent with added negative selection pressure in the form of non-target cells promoting retrieval of binders with increasing selectivity for target compared with non-target cells and reducing binders specific for commonly expressed and target cell weakly upregulated receptors, as modelled in silico (FIG. 3) and experimentally verified by flow-cytometry analyses (FIG. 4).

Extending the comparative analysis to include the total 94,429 sequences indicated by enrichment signatures to encode antibodies to low expressed restricted, or upregulated, receptors (FIG. 7A, lower group), allowed a fraction of these antibodies to be classified into either category. Accordingly, 13,709 antibodies specific for low expressed, restricted receptors (increasing frequency), and 753 antibodies to upregulated receptors (decreasing frequency) were indicated (FIG. 8B). Remaining antibodies were less clearly affected by competition selection (FIG. 8B; n=79,967).

### Example 6—Predictions Discover Unparalleled Numbers of Antibodies with Distinct Therapeutic and Diagnostic Potential

The collective data thus indicated that prediction-guided selections had generated a highly diversified pool of antibodies to a priori unknown, differentially expressed, surface receptors expressed over a wide dynamic range, and that enrichment signatures could be used to identify unparalleled numbers (˜10⁵ compared with ˜10³) of antibodies to therapeutically and diagnostically relevant receptors. To address its relevance for such antibody discovery, a subset of antibody sequences (n=10²) indicated by enrichment signatures to encode antibodies specific for receptors expressed at (Group 1)>10⁶ molecules/cell (n=8), (Group 2) 10⁵ to 10⁶ molecules/cell (n=16), (Group 3)<10⁵ molecules/cell or target cell upregulated receptors (n=67), or (Group 4) antibodies to receptors<5-fold upregulated on target cells or lacking specificity for target cell receptors altogether (n=11) were selected for production in full-length IgG format (FIG. 9, upper panel). After expression and purification, the number of antibody epitopes (i.e. the expression of targeted receptors on target and non-target cells) were quantified by flow-cytometry using ABC beads (FIG. 9, lower panel). Consistent with the ability of the method of the present invention to generate unparalleled numbers of antibodies to differentially expressed surface receptors, 93% (85/91) of the antibodies in Groups 1-3 were confirmed to be specific to differentially expressed receptors on target cells. Equally important, and demonstrating the value of the prediction-guided approach, 9/11 (82%) antibodies with apparent enrichment between selection rounds 1 and 2, but indicated by enrichment signatures to lack specificity for medically relevant receptors, were confirmed negative for binding to target cells.

Furthermore, and consistent with enrichment signatures informing antibody specificity, experimentally determined numbers of antibody-targeted receptors on target cells correlated well with those predicted. Antibodies in Group 1 were predicted to bind receptors with >10⁶ copies/cell, and the experimentally determined target cell expression was 1.4×10⁶ (7.3×10⁵ to 2.6×10⁶) (geometric mean (95% confidence interval)). Group 2 was predicted to bind receptors with expression levels between 10⁵ and 10⁶ and the experimentally determined number was 3.6×10⁵ (1.0×10⁵ to 1.3×10⁶). Finally, Group 3 was predicted to bind restricted receptors with expression levels<10⁵, or upregulated receptors.

To help separate these different categories of antibodies, we performed comparative analyses of Group 3 enrichment signatures from selections with or without non-target cell competition. This data set contained sequences that showed either increasing (28/67) or decreasing (7/67) frequency in the presence compared to the absence of competition selection (FIG. 9, inset). Consistent with these respective enrichment signatures informing antibody specificity for upregulated and restricted low expressed receptors, respectively, the experimentally determined number of epitopes on target cells and non-target cells for antibodies with predicted specificity for upregulated receptors were 5.4×105 (1.4×105 to 2.2×106) and 1.3×104 (2.3×10³ to 7.9×104), and for antibodies with predicted specificity for restricted, low expressed receptors, 3.0×104 (1.3×104 to 7.1×104) and non-determinable, the latter since 22/28 antibodies in this group bound receptors with non-target cell expression below the detection limit (detection limit=1×103 receptors/cell (FIG. 9, inset).

While, overall, antibody specificity could be predicted by the enrichment signatures, the specificity of some individual antibodies deviated from predictions (FIG. 9). For example, only Group 1 (left hand panels) was predicted to contain antibodies specific for receptors expressed at >1 million copies/target cell with no expression on non-target cells. However, all groups contained a fraction of antibodies with this expression profile. Since the modeling data indicated differential enrichment of antibodies with varying affinity to the same receptor (or receptors with the same expression level) and naïve antibody libraries contain antibodies of varying affinity, we next analysed how antibody affinity affected antibody enrichment signatures. Antibodies with similar determined epitopes (i.e. similar receptor expression levels on target cells) were divided in two groups according to their >3 nM or <3 nM EC₅₀ values for binding to endogenously expressed receptors and their enrichment during selection was compared. Consistent with the modeling shown in FIG. 6A, it was found that for both monitored target expression levels (1-4 million copies per cell, n=22 and 300.000-1 million copies per cell, n=10), the frequency of antibodies with an EC₅₀ value<3 nM were higher than the frequency of antibodies with an EC₅₀ value>3 nM. For antibodies to receptors with 1-4 million copies per cell, this difference was statistically significant (p<0.05) after selections 2, 3, and 4 (FIG. 8C).

In summary, the method of the present invention enabled discovery of unparalleled numbers of antibodies to differentially expressed surface receptors of distinct potential therapeutic or diagnostic value.

### Discussion of the Results in Examples 3 to 6

This study describes the use of the novel selection and screening methodology, which enables rational identification of antibodies to a priori unknown, differentially expressed, cell surface receptors. Compared with previously described technologies, this method identified orders of magnitude greater numbers of antibodies, which were shown to bind receptors expressed over a wide dynamic range, including low expressed, restricted receptors. As such, prediction-based discovery overcomes several limitations of selection and screening methodology in the art.

The method of the present invention is of particular use in relation to in phenotypic discovery (PD) of biologics (for a review on PD see Moffat et al, 2017). In PDD, small molecules or antibodies from large molecular libraries are screened for functional activity (e.g. inhibition of pro-inflammatory cell cytokine release or induction of tumor cell death) without prior knowledge of their molecular targets. Consequently, PDD enables discovery of the most functional molecules and antibodies across multiple receptors and epitopes for specific disease-associated pathways. PDD is a well-validated strategy for first-in-class small molecule drug discovery (Swinney, 2013; Swinney and Lee, 2020), and has been used to identify several first-in-class antibodies and their associated targets e.g. CD52 (Waldmann et al, 1984), ICAM-1 (Veitonmaki et al, 2013), CD32b (Roghanian et al, 2015; Ljungars et al, 2018), TNFR2 (Williams et al, 2016) that are currently in clinical development. By combining the ability of prediction-based discovery to identify a large number of antibodies to a broad range of surface receptors, with appropriate functional screening (Ljungars et al, 2018; Chandrasekaran et al, 2021) and a recently described high-throughput CRISPR based method for target deconvolution (Mattsson et al, 2021), biologics PDD can be taken to the next level—directly on par with small molecule PDD.

A key feature of the method of the present invention, relevant to both diagnostic and therapeutic applications, is its ability to identify antibodies to low expressed disease-associated molecules. Antibodies to low expressed tumor-restricted antigens and rare disease-associated configurational epitopes may have significant therapeutic potential when developed as empowered biologics. Concerning diagnostics, liquid biopsies can be easily sampled, but typically contain biomarkers in very low concentrations. As such, technologies that help identify and quantitate rare disease-associated biomarkers are instrumental to the pursuit of earlier diagnosis and personalised medicine. The observations in this study that antibodies to low expressed and upregulated receptors are present at very low frequency (one or fewer clones per million) in selected pools are consistent with the observed shortcoming of existing methods to generate such antibodies. In the absence of integrated computational modeling and informed enrichment signatures identified in data generated by massively parallel sequencing, robust identification of these specificities would require production and labor-intensive cell-based screening of millions of antibody clones. Antibodies to low expressed receptors are also of value in diagnostics.

Although in this study prediction-based discovery was applied to identify antibodies that discriminate one cell type from another, it is broadly applicable to other complex antigen systems e.g. blood, urine, cerebrospinal fluid (Cortese et al, 1996), tissue (Larsen et al, 2015), bacteria (DiGiandomenico et al, 2012), or viruses (van der Brink et al, 2005) relevant to diverse inflammatory, immunological, neurological, infectious diseases and cancer. Another highly relevant application of the present invention is to identify antibodies to key virulence factors, e.g. adhesive glycoproteins of pandemic microorganisms (Bertoglio et al, 2021) and their receptors on host cells. Identification of such antibodies and their associated molecular targets has the potential to generate both passive (antibody-based) and active (vaccine) immunotherapies to help treat and prevent drug-resistant microbial infections.

A further advantage of prediction-based discovery, relative to other discovery methodologies (e.g. gene expression-based or proteomics-based approaches) is the parallel discovery of target molecules and of candidate therapeutic or diagnostic antibodies against these targets, which may be of composite or processed nature, e.g. oxidised low-density lipoprotein (Schiopu et al, 2007; Lehrer-Graiwer et al, 2015; Li et al, 2013), and may include disease-associated epitopes and configurations.

Key applications underlying the ability of prediction-based discovery to generate comprehensive panels of binders to differentially expressed molecules, relate to the integration of in silico modeling and experimental selection. Firstly, in silico modeling is utilised to establish selection reaction parameters needed to achieve robust enrichment of relevant binders, and deselection of trivial binders, without knowing actual binder specificities, or targeted molecules' abundance. This is achieved by simulating enrichment of hypothetical binders-defined by their specificity for molecules with different absolute and relative expression levels in two complex antigen populations e.g. diseased compared with healthy blood, serum or cells. As modelled and experimentally verified in this study, insufficient positive selection pressure will result in fewer retained binders, and a risk of depleting binders to lower expressed molecules (data not shown). Insufficient subtractive selection pressure, on the other hand, generates binders to the most highly expressed, rather than the strongest differentially expressed (i.e. disease-associated molecules) (FIG. 2). Retrospective application of in silico modeling to previously described differential selection protocols, is consistent with both insufficient positive and subtractive selection pressure underlying poor observed binder qualities and quantities (data not shown).

Secondly, the method of the present invention enables unparalleled discovery of binders with distinct therapeutic and diagnostic potential, by integrating in silico modelling with experimental differential selection. Specifically, the fraction of binders that has been enriched in an antigen-specific manner (i.e. the binder hit-rate), is experimentally determined. Enrichment signatures of categorised binders, defined as above by their specificity for molecules with different absolute and relative expression levels in two antigen populations, are then generated by in silico modeling of the antigen-specific enriched binder fraction according to targeted molecules abundance, and presence or absence of competition selection. By matching enrichment signatures of individual experimentally identified binder sequences to in silico modelled reference enrichment signatures, binders to some differentially expressed molecules can be identified with high precision (90%) in unparalleled numbers (˜100,000). This same methodology allows for informed prioritisation of binders according to indicated therapeutic and diagnostic use (e.g. naked or empowered IgG).

In this study, presented equations were used to in silico optimise selection reactions, and to generate predicted reference enrichment signatures for identification of (experimentally enriched) antibody sequences encoding specificity for therapeutically and diagnostically relevant cell surface receptors. Modelling can, however, also be used to model antibody enrichment of already performed selections. Retrospective application of in silico modeling to ours and others previously described differential selection protocols, is consistent with both insufficient positive and subtractive selection pressure underlying limited observed antibody qualities and quantities. As such, predictions may help “rescue” existing antibody pools generated using precious patient-derived materials, by re-analyses incorporating enrichment signatures, and/or by indicating benefit of performing additional (optimised) selection rounds in presence or absence of competition.

In conclusion, and with special reference to PDD, prediction-based discovery shifts the current bottle neck from identification of a large number of antibodies to sum differentially expressed, disease-associated, biomolecules, to the development of efficient antibody production and high-throughput clinically predictive functional assays, which allow for screening of (tens of) thousands of clones.

### REFERENCES

Barbas C F, Kang A S, Lerner R A and Benkovic S J, (1991), Assembly of combinatorial antibody libraries on phage surfaces: the gene III site. Proc. Natl. Acad. Sci. USA 88, 7978-7982.
Barreto, K. et al. (2019). Next-generation sequencing-guided identification and reconstruction of antibody CDR combinations from phage selection outputs. Nucleic acids research, 47 (9): e50.
Beck et al. (2010). Strategies and challenges for the next generation of therapeutic antibodies. Nat Rev Immunol 10, 345-352.
Beers et al. (2008). Type II (tositumomab) anti-CD20 monoclonal antibody out performs type I (rituximab-like) reagents in B-cell depletion regardless of complement activation. Blood 112, 4170-4177.
Bentley D R, et al. Accurate whole human genome sequencing using reversible terminator chemistry. Nature. 2008; 456:53-59.
Bertoglio, F. et al. (2021). SARS-COV-2 neutralizing human recombinant antibodies selected from pre-pandemic healthy donors binding at RBD-ACE2 interface. Nat Commun 12, 1577.
Boder and Wittrup (1997). Yeast surface display for screening combinatorial polypeptide libraries. Nat Biotechnol 15, 553-557.
Carlsson, R. et al. (1988) Binding of staphylococcal enterotoxin A to accessory cells is a requirement for its ability to activate human T cells. J Immunol 140, 2484.
Chandrasekaran, S. N., Ceulemans, H., Boyd, J. D. & Carpenter, A. E. (2021). Image-based profiling for drug discovery: due for a machine-learning upgrade? Nat Rev Drug Discov, 20, 145-159.
Chiswell D J and McCafferty J, (1992), Phage antibodies: will new ‘coliclonal’ antibodies replace monoclonal antibodies? Trends Biotechnol. 10, 80-84.
Clackson T, Hoogenboom H R, Griffiths A D and Winter G, (1991), Making antibody fragments using phage display libraries. Nature 352, 624-628.
Cortese, I. et al. (1996). Identification of peptides specific for cerebrospinal fluid antibodies in multiple sclerosis by using phage libraries. Proc Natl Acad Sci USA 93, 11063-11067.
Cragg, M. S., and Glennie, M. J. (2004). Antibody specificity controls in vivo effector mechanisms of anti-CD20 reagents. Blood 103, 2738-2743.
de Kruif, J., Terstappen, L., Boel, E. & Logtenberg, T. (1995). Rapid selection of cell subpopulation-specific human monoclonal antibodies from a synthetic phage antibody library. Proc Natl Acad Sci USA 92, 3938-3942.
DiGiandomenico, A. et al. (2012). Identification of broadly protective human antibodies to Pseudomonas aeruginosa exopolysaccharide Psl by phenotypic screening. J Exp Med 209, 1273-1287.
Dower, W. J., CWIRLA, S. E. and N. V., A. T. (1991) Recombinant Library Screening Methods. In: World Intellectual Property Organization. SMITH, W. M., United States.
Drmanac R, et al. Human Genome Sequencing Using Unchained Base Reads on Self-assembing DNA Nanoarrays. Science 2010; 327:78-81.
Dyer, M. J., Hale, G., Hayhoe, F. G. & Waldmann, H. (1989). Effects of CAMPATH-1 antibodies in vivo in patients with lymphoid malignancies: influence of antibody isotype. Blood 73, 1431-1439.
Egloff et al (2019). Engineered peptide barcodes for in-depth analyses of binding protein libraries. Nat Methods 16, 421-428.
Ellington and Szostak (1990). In vitro selection of RNA molecules that bind specific ligands. Nature 346, 818-822.
Felici F, Luzzago A, Manaci P, Nicosia A, Sollazzo M and Traboni C, (1995), Peptide and protein display on the surface of filamentous bacteriophage. Biotechnol. Annual Rev. 1, 149-183.
Francisco, J. A., Stathopoulos, C., Warren, R. A., Kilburn, D. G. and Georgiou, G. (1993) Specific adhesion and hydrolysis of cellulose by intact Escherichia coli expressing surface anchored cellulase or cellulose binding domains. Biotechnology (N Y) 11, 491.
Fransson, J., Tornberg, U. C., Borrebaeck, C. A., Carlsson, R., and Frendeus, B. (2006). Rapid induction of apoptosis in B-cell lymphoma by functionally isolated human antibodies. Int J Cancer 119, 349-358.
Frendeus, B. (2013). Function-first antibody discovery: Embracing the unpredictable biology of antibodies. Oncoimmunology 2, e25047.
Gao, C., Lin, C. H., Lo, C. H., Mao, S., Wirsching, P., Lerner, R. A. and Janda, K. D. (1997) Making chemistry selectable by linking it to infectivity. Proc Natl Acad Sci USA 94, 11777.
Hanes, J. and Pluckthun, A. (1997) In vitro selection and evolution of functional proteins by using ribosome display. Proc Natl Acad Sci USA 94, 4937.
Hanes, J., Schaffitzel, C., Knappik, A. & Pluckthun (2000). A. Picomolar affinity antibodies from a fully synthetic naive library selected and evolved by ribosome display. Nat Biotechnol 18, 1287-1292.
Harris T D, et al. (2008). Single-molecule DNA sequencing of a viral genome. Science; 320:106-109.
He, M. and Taussig, M. J. (1997) Antibody-ribosome-mRNA (ARM) complexes as efficient selection particles for in vitro display and evolution of antibody combining sites. Nucleic Acids Res 25, 5132.
Hoogenboom H R and Winter G, (1992), By-passing immunisation. Human antibodies from synthetic repertoires of germline VH gene segments rearranged in vitro. J. Mol. Biol. 227, 381-388).
Hoogenboom H R, deBruine A P, Hutton S E, Hoet R M, Arends J W and Rooves R C, (1998), Antibody phage display technology and its application. Immunotechnology 4 (1), 1-20.
Hoogenboom, H. R. (2002). Overview of antibody phage display technology and its applications. In Methods in Molecular Biology P. M. O'Brien, and R. Aitken, eds. (Totowa, NJ: Humana Press Inc.), pp. 1-37.
Jacobsson, K. and Frykberg, L. (1995) Cloning of ligand-binding domains of bacterial receptors by phage display. Biotechniques 18, 878.
Katz B A, (1997), Structural and mechanistic determinants of affinity and specificity of ligands discovered or engineered by peptide display. Annual Rev. Biophys. Biomol. Struct. 26, 27-45
Kay B K and Paul J I, (1996), High-throughput screening strategies to identify inhibitors of protein-protein interactions. Mol. Divers. 1, 139-140).
Koide, A., Bailey, C. W., Huang, X. and Koide, S. (1998) The fibronectin type III domain as a scaffold for novel binding proteins. J Mol Biol 284, 1141.
Larsen, S. A., Meldgaard, T., Lykkemark, S., Mandrup, O. A. & Kristensen, P. (2015). Selection of cell-type specific antibodies on tissue-sections using phage display. J Cell Mol Med 19, 1939-1948.
Lehrer-Graiwer, J. et al. (2015). FDG-PET imaging for oxidized LDL in stable atherosclerotic disease: a phase II study of safety, tolerability, and anti-inflammatory activity. JACC Cardiovasc Imaging 8, 493-494.
Li, S. et al. (2013). Targeting oxidized LDL improves insulin sensitivity and immune cell function in obese Rhesus macaques. Mol Metab 2, 256-269.
Liu et al. (2000). Differential expression of cell surface molecules in prostate cancer cells. Cancer research; 60, 3429-3434.
Liu et al. (2004). Mapping tumor epitope space by direct selection of single-chain Fv antibody libraries on prostate cancer cells. Cancer Res 64, 704-710.
Ljungars et al. (2018). A platform for phenotypic discovery of therapeutic antibodies and targets applied on Chronic Lymphocytic Leukemia. NPJ Precision Oncology; 2, 18.
Ljungars et al. (2019). Deep Mining of Complex Antibody Phage Pools Generated by Cell Panning Enables Discovery of Rare Antibodies Binding New Targets and Epitopes. Frontiers in Pharmacology; 10, 847.
Lopez, T., Nam, D. H., Kaihara, E., Mustafa, Z. & Ge, X. (2017). Identification of highly selective MMP-14 inhibitory Fabs by deep sequencing. Biotechnology and bioengineering 114, 1140-1150.
Lundquist P M, et al. (2008). Parallel confocal detection of single molecules in real time. Optics Letters 33:1026-1028.
Margulies M, et al. (2005). Genome sequencing in microfabricated high-density picolitre reactors. Nature; 437:376-380.
Markland, W., Roberts, B. L., Saxena, M. J., Guterman, S. K. and Ladner, R. C. (1991) Design, construction and function of a multicopy display vector using fusions to the major coat protein of bacteriophage M13. Gene 109, 13.
Marks J D, Hoogenboom H R, Bonnert T P, McCafferty J, Griffiths A D and Winter G, (1991), By-passing immunisation. Human antibodies from V-gene libraries displayed on phage. J. Mol. Biol. 222, 581-597
Mattheakis, L. C., Bhatt, R. R. and Dower, W. J. (1994) An in vitro polysome display system for identifying ligands from very large peptide libraries. Proc Natl Acad Sci USA 91, 9022.
Mattsson, J. et al. (2021). Accelerating target deconvolution for therapeutic antibody candidates using highly parallelized genome editing. Nat Commun 12, 1277.
McCafferty, J., Griffiths, A. D., Winter, G. and Chiswell, D. J. (1990) Phage antibodies: filamentous phage displaying antibody variable domains. Nature 348, 552.
Moffat, J. G., Vincent, F., Lee, J. A., Eder, J. & Prunotto, M. (2017). Opportunities and challenges in phenotypic drug discovery: an industry perspective. Nat Rev Drug Discov 16, 531-543.
Mutuberria et al. (1999). Model systems to study the parameters determining the success of phage antibody selections on complex antigens. J Immunol Methods 231, 65-81.
Nixon et al (2019). A rapid in vitro methodology for simultaneous target discovery and antibody generation against functional cell subpopulations. Sci Rep 9, 842
Osbourn et al. (1998). Pathfinder selection: in situ isolation of novel antibodies. Immunotechnology 3, 293-302.
Parmley and Smith (1988). Antibody-selectable filamentous fd phage vectors: affinity purification of target genes. Gene, 73 (2): 305-318.
Qin, H. et al. (2014). Generation of a new therapeutic peptide that depletes myeloid-derived suppressor cells in tumor-bearing mice. Nat Med 20, 676-681.
Ridgway, J. B. et al. (1999). Identification of a human anti-CD55 single-chain Fv by subtractive panning of a phage library using tumor and nontumor cell lines. Cancer Res 59, 2718-2723.
Roghanian, A. et al. (2015). Antagonistic human FcgammaRIIB (CD32B) antibodies have anti-tumor activity and overcome resistance to antibody therapy in vivo. Cancer Cell 27, 473-488.
Rothe, C. et al. (2008). The human combinatorial antibody library HuCAL GOLD combines diversification of all six CDRs according to the natural immune system with a novel display method for efficient selection of high-affinity antibodies. J Mol Biol 376, 1182-1200.
Sandercock et al (2015). Identification of anti-tumour biologics using primary tumour models, 3-D phenotypic screening and image-based multi-parametric profiling. Mol Cancer 14, 147
Schiopu, A. et al. (2007). Recombinant antibodies to an oxidized low-density lipoprotein epitope induce rapid regression of atherosclerosis in apobec-1 (−/−)/low-density lipoprotein receptor (−/−) mice. J Am Coll Cardiol 50, 2313-2318.
Sebba, A. (2008). Tocilizumab: the first interleukin-6-receptor inhibitor. Am J Health Syst Pharm 65, 1413-1418.
Semmrich et al (2022). Vectorized Treg-depleting aCTLA-4 elicits antigen cross-presentation and CD8 (+) T cell immunity to reject ‘cold’ tumors. J Immunother Cancer 10.
Shendure J, et al. (2005). Accurate multiplex polony sequencing of an evolved bacterial genome. Science; 309:1728-1732.
Siegel et al. (1997). Isolation of cell surface-specific human monoclonal antibodies using phage display and magnetically-activated cell sorting: applications in immunohematology. J Immunol Methods 206, 73-85
Smith, G. P. (1985) Filamentous fusion phage: novel expression vectors that display cloned antigens on the virion surface. Science 228, 1315.
Smith, G. P. and Scott, J. K. (1993) Libraries of peptides and proteins displayed on filamentous phage. Methods Enzymol 217, 228.
Soderlind et al. (2000). Recombining germline-derived CDR sequences for creating diverse single-framework antibody libraries. Nat Biotechnol 18, 852-856.
Stahl et al. (1989) A dual expression system for the generation, analysis and purification of antibodies to a repeated sequence of the Plasmodium falciparum antigen Pf155/RESA. J Immunol Methods 124, 43.
Swinney, D. C. (2013). Phenotypic vs. target-based drug discovery for first-in-class medicines. Clin Pharmacol Ther 93, 299-301.
Swinney, D. C. & Lee, J. A. (2020). Recent advances in phenotypic drug discovery. F1000Res 9.
van den Brink, E. N. et al. (2005). Molecular and biological characterization of human monoclonal antibodies binding to the spike and nucleocapsid proteins of severe acute respiratory syndrome coronavirus. J Virol 79, 1635-1644.
Veitonmaki, N. et al. (2013). A human ICAM-1 antibody isolated by a function-first approach has potent macrophage-dependent antimyeloma activity in vivo. Cancer Cell 23, 502-515.
Waldmann, H. et al. (1984). Elimination of graft-versus-host disease by in-vitro depletion of alloreactive lymphocytes with a monoclonal rat anti-human lymphocyte antibody (CAMPATH-1). Lancet 2, 483-486.
Weng, S., Gu, K., Hammond, P. W., Lohse, P., Rise, C., Wagner, R. W., Wright, M. C. and Kuimelis, R. G. (2002) Generating addressable protein microarrays with PROfusion covalent mRNA-protein fusion technology. Proteomics 2, 48.
Williams, B. R. and Sharon, J. (2002) Polyclonal anti-colorectal cancer Fab phage display library selected in one round using density gradient centrifugation to separate antigen-bound and free phage. Immunol Lett 81, 141.
Williams, G. S. et al. (2016). Phenotypic screening reveals TNFR2 as a promising target for cancer immunotherapy. Oncotarget 7, 68278-68291.
Winter and McCafferty (1996) Phage display of peptides and proteins: a laboratory manual Ed Kay, Academic Press, Inc ISBN 0-12-402380-0
Winter, G., Griffiths, A. D., Hawkins, R. E. and Hoogenboom, H. R. (1994) Making antibodies by phage display technology. Annu Rev Immunol 12, 433.

Embodiments of the invention will now be described in the following numbered paragraphs:
1. A method of isolating from a library of anti-ligands at least one anti-ligand to at least one differentially-expressed target ligand in a target cell, tissue, or sample of interest, wherein the method comprises the steps of:
   (a) providing one or more reference enrichment signature for the library of anti-ligands used;
   (b) performing one or more round of differential biopanning on the library of anti-ligands to produce anti-ligand pools;
   (c) performing high throughput sequencing on the anti-ligand pools produced during step (b), so as to generate a discovery enrichment signature for each anti-ligand in the anti-ligand pools.
2. The method according to paragraph 1, wherein the method further comprises:
   (d) matching the one or more reference enrichment signature provided in step (a) with a discovery enrichment signature for an anti-ligand generated in step (c), so as to isolate at least one anti-ligand to at least one differentially-expressed target ligand in a target cell, tissue, or sample of interest.
3. The method according to paragraph 1 or paragraph 2, wherein the one or more reference enrichment signature provided in step (a) is an in silico-derived reference enrichment.
4. The method according to paragraph 3, wherein the in silico-derived reference enrichment signature is generated using an equation derived from the universal law of mass of action, optionally wherein the in silico-derived signature is generated using the equation:

$${FA}_{T} = \frac{{rA}_{T}}{( {\sum }_{1}^{n} {rA}_{T} )} \times HR$$

where
FA_{T}=frequency of recovered antibody
rA_{T}=number of recovered antibodies

$${\sum }_{1}^{n} {rA}_{T} = Total number of recovered antibodies$$

HR=hit-rate, fraction of antibodies specific for the target cells, tissues, or samples (experimentally determined).
5. The method according to paragraph 3 or paragraph 4, wherein the in silico-derived reference enrichment signature is for an anti-ligand to a high expressed ligand of a target cell, tissue, or sample of interest, for an anti-ligand to an intermediary expressed ligand of a target cell, tissue, or sample of interest; or for an anti-ligand to a low expressed ligand of a target cell, tissue, or sample of interest.
6. The method according to paragraph 1 or paragraph 2, wherein the one or more reference enrichment signature provided in step (a) is an experimentally-derived reference enrichment signature.
7. The method according to paragraph 6, wherein the experimentally-derived reference enrichment signature is generated from at least one biopanning experiment comprising at least one reference anti-ligand.
8. The method according to paragraph 6 or paragraph 7, wherein the experimentally-derived reference enrichment signature is for an anti-ligand to a high expressed ligand of a target cell, tissue, or sample of interest, for an anti-ligand to an intermediary expressed ligand of a target cell, tissue, or sample of interest; or for an anti-ligand to a low expressed ligand of a target cell, tissue, or sample of interest.
9. The method according to paragraph 1 or paragraph 2, wherein in step (a) two or more reference enrichment signatures are provided, wherein one or more of the reference enrichment signatures is an in silico-derived reference enrichment signature, and wherein one or more of the reference enrichment signatures is an experimentally-derived enrichment signature.
10. The method according to paragraph 9, wherein the in silico-derived reference enrichment signature is generated using an equation derived from the universal law of mass of action, optionally wherein the in silico-derived enrichment signature is generated using the equation:

$${FA}_{T} = \frac{{rA}_{T}}{( {\sum }_{1}^{n} {rA}_{T} )} \times HR$$

where
FA_{T}=frequency of recovered antibody
rA_{T}=number of recovered antibodies

$${\sum }_{1}^{n} {rA}_{T} = Total number of recovered antibodies$$

HR=hit-rate, fraction of antibodies specific for the target cells, tissues, or samples (experimentally determined).
11. The method according to paragraph 9 or paragraph 10, wherein the experimentally-derived reference enrichment signature is generated from at least one biopanning experiment comprising at least one reference anti-ligand.
12. The method according to any one of paragraphs 9 to 11, wherein the in silico-derived reference enrichment signature is for an anti-ligand to a high expressed ligand of a target cell, tissue, or sample of interest, for an anti-ligand to an intermediary expressed ligand of a target cell, tissue, or sample of interest; or for an anti-ligand to a low expressed ligand of a target cell, tissue, or sample of interest, and/or
   wherein the experimentally-derived reference enrichment signature is for an anti-ligand to a high expressed ligand of a target cell, tissue, or sample of interest, for an anti-ligand to an intermediary expressed ligand of a target cell, tissue, or sample of interest; or for an anti-ligand to a low expressed ligand of a target cell, tissue, or sample of interest.
13. The method according to any one of paragraphs 1 to 12, wherein biopanning step (a) comprises the sub-steps of:
   (i) providing a library of anti-ligands;
   (ii) providing a first population of ligands comprising a ligand fixed to or incorporated in a subtractor ligand construct;
   (iii) providing a second population of ligands comprising a ligand fixed to or incorporated in a target ligand construct;
   (iv) determining an amount of the subtractor ligand construct and the target ligand construct in the populations using one or more equations derived from the universal law of mass action

$$\frac{{{[ C ]}^{c} [ D ]}^{d}}{{{[ A ]}^{a} [ B ]}^{b}} = {K}_{eq} ,$$

where:
A, B, C & D=are the participants in the reaction (reactants and products)
a, b, c, & d=the coefficients necessary for a balanced chemical equation
so as to permit isolation of anti-ligand to differentially expressed target ligand;
(v) providing the amount of subtractor ligand construct as determined in step (iv);
(vi) providing the amount of target ligand construct as determined in step (iv);
(vii) providing separation means for isolating anti-ligand bound to the target ligand construct from anti-ligand bound to a subtractor ligand construct;
(viii) exposing the library of (i) to the ligand constructs provided by (v) and (vi) to permit binding of anti-ligands to ligands; and
(ix) using the separation means to isolate anti-ligand bound to the ligand fixed to or incorporated in the target ligand construct.
14. The method according to paragraph 13, wherein the equation in step (iv) is either:

$$bA = \frac{A + B + {K}_{d} {N}_{A} V}{2} - \sqrt{\frac{{( A + B + {K}_{d} {N}_{A} V )}^{2}}{4} - AB}$$

where
bA=number of receptor bound anti-ligands
A=total number of anti-ligands A
B=total number of target receptors B
K_{d}=Anti-ligand affinity (M)
V=the reaction volume (dm³)
N_{A}=Avogadro's constant (6.022×10²³ particles mole⁻¹)
or

$${bA}_{T} = ( \frac{A + B + {K}_{d} {N}_{A} V}{2} - \sqrt{\frac{{( A + B + {K}_{d} {N}_{A} V )}^{2}}{4} - AB} ) \times ( \frac{{B}_{T} {C}_{T}}{{B}_{T} {C}_{T} + {B}_{N} {C}_{N}} ) \qquad ( 5 )$$

where
bA=number of receptor bound anti-ligands
A=total number of anti-ligands A
B=total number of target receptors B
K_{d}=Anti-ligand affinity (M)
V=the reaction volume (dm³)
N_{A}=Avogadro's constant (6.022×10²³ particles mole⁻¹)
C_{T}=the number of target cells
C_{N}=the number of non-target cells
B_{T}=the number of receptors on C_{T}
B_{N}=the number of receptors on C_{N}.
15. The method according to any one of paragraphs 1 to 14, wherein step (b) comprises performing two or more rounds of biopanning, three or more rounds of biopanning, or four or more rounds of biopanning.
16. The method according to any one of paragraphs 1 to 15, wherein the differentially expressed ligand is:
   a high expressed ligand of a target cell, tissue, or sample of interest;
   an intermediary expressed ligand of a target cell, tissue, or sample of interest; or,
   a low expressed ligand of a target cell, tissue, or sample of interest.
17. The method according to any one of paragraphs 13 to 16, wherein the ligand is not expressed on one of either the target construct or the subtractor construct.
18. The method according to any one of paragraphs 1 to 17, wherein the method further comprises a step of releasing the anti-ligand from the ligand.
19. The method according to any one of paragraphs 13 to 18, wherein steps (ii) to (ix) are conducted in parallel to isolate a plurality of anti-ligands to a plurality of different ligands; and/or wherein steps (ii) to (ix) are repeated one or more times.
20. The method according to any one of paragraphs 13 to 19, wherein the amount of one of the subtractor construct or target construct is provided in excess of the amount of the other of the subtractor construct or target construct; and/or wherein the excess of ligand is between 10 and 1000 fold, or 2 and 10 fold, or 1000 and 1,000,000 fold.
21. The method according to any one of paragraphs 1 to 20, wherein the high throughput sequencing in step (c) is carried out using 454 sequencing, Illumina, SOLID methods or the Helicos system.
22. The method according to any one of paragraphs 13 to 21, wherein the separation means are selected from at least one of a solid support, cell membrane and/or portions thereof, synthetic membrane, beads, chemical tags and free ligand, or fluorescence activated cell sorting.
23. The method according to any one of paragraphs 13 to 22, wherein step (ix) is performed by at least one of density centrifugation, solid support sequestration, magnetic bead sequestration, chemical tag binding and aqueous phase partitioning, fluorescent activated cell sorting.
24. The method according to any one of paragraphs 1 to 23, wherein the library of anti-ligands is a display library comprising a plurality of library members which display anti-ligands; optionally wherein the library is a phage display library and/or wherein the library of anti-ligands is constructed from at least one of antibodies, antigen binding variants, derivatives and/or fragments thereof; scaffold molecules with engineered variable surfaces; receptors; and enzymes.
25. The method according to any one of paragraphs 1 to 24, wherein the library of anti-ligands is a pool of anti-ligands generated by a previously conducted anti-ligand library selection experiment.
26. The method according to any one of paragraphs 1 to 25, wherein the library of anti-ligands comprises at least one anti-ligand having a known copy number, optionally wherein the at least one anti-ligand having a known copy number is added to the library of anti-ligands at a known copy number.
27. The method according to any one of paragraphs 1 to 26, wherein the ligand is at least one selected from antigens; receptor ligands; and enzyme targets that comprise at least one of carbohydrate; protein; peptide; lipid; polynucleotide; inorganic molecules and conjugated molecules; optionally wherein the ligand is a cell surface receptor, optionally wherein the cell surface receptor is in its native form.
28. A method of isolating from a library of anti-ligands at least one anti-ligand to at least one differentially-expressed target ligand in a target cell, tissue, or sample of interest, wherein the method comprises the steps of:
   (a) providing one or more reference enrichment signature for an anti-ligand present in the library of anti-ligands used; and,
   (b) providing one or more discovery enrichment signature for an anti-ligand present in the library of anti-ligands used.
29. The method according to paragraph 28, wherein the method further comprises:
   (c) matching the one or more reference enrichment signature provided in step (a) with the one or more discovery enrichment signature provided in step (b), so as to isolate at least one anti-ligand to at least one differentially-expressed target ligand in a target cell, tissue, or sample of interest.
30. The method according to paragraph 28 or paragraph 29, wherein the one or more reference enrichment signature provided in step (a) is an in silico-derived reference enrichment.
31. The method according to paragraph 30, wherein the in silico-derived reference enrichment signature is generated using an equation derived from the universal law of mass of action, optionally wherein the in silico-derived enrichment signature is generated using the equation:

$${FA}_{T} = \frac{{rA}_{T}}{( {\sum }_{1}^{n} {rA}_{T} )} \times HR$$

where
FA_{T}=frequency of recovered antibody
rA_{T}=number of recovered antibodies

$${\sum }_{1}^{n} {rA}_{T} = Total number of recovered antibodies$$

HR=hit-rate, fraction of antibodies specific for the target cells, tissues, or samples (experimentally determined),
optionally wherein the in silico-derived reference enrichment signature is for an anti-ligand to a high expressed ligand of a target cell, tissue, or sample of interest, for an anti-ligand to an intermediary expressed ligand of a target cell, tissue, or sample of interest; or for an anti-ligand to a low expressed ligand of a target cell, tissue, or sample of interest.
32. The method according to paragraph 28 or paragraph 29, wherein the one or more reference enrichment signature provided in step (a) is an experimentally-derived reference enrichment signature.
33. The method according to paragraph 32, wherein the experimentally-derived reference enrichment signature is generated from at least one biopanning experiment comprising at least one reference anti-ligand.
34. The method according to paragraph 33, wherein the experimentally-derived reference enrichment signature is for an anti-ligand to a high expressed ligand of a target cell, tissue, or sample of interest, for an anti-ligand to an intermediary expressed ligand of a target cell, tissue, or sample of interest; or for an anti-ligand to a low expressed ligand of a target cell, tissue, or sample of interest.
35. The method according to paragraph 28 or paragraph 29, wherein in step (a) two or more reference enrichment signatures are provided, wherein one or more of the reference enrichment signatures is an in silico-derived reference enrichment signature, and wherein one or more of the reference enrichment signatures is an experimentally-derived enrichment signature.
36. The method according to paragraph 35, wherein the in silico-derived reference enrichment signature is generated using an equation derived from the universal law of mass of action, optionally wherein the in silico-derived enrichment signature is generated using the equation:

$${FA}_{T} = \frac{{rA}_{T}}{( {\sum }_{1}^{n} {rA}_{T} )} \times HR$$

where
FA_{T}=frequency of recovered antibody
rA_{T}=number of recovered antibodies

$${\sum }_{1}^{n} {rA}_{T} = Total number of recovered antibodies$$

HR=hit-rate, fraction of antibodies specific for the target cells, tissues, or samples (experimentally determined).
37. The method according to paragraph 35 or paragraph 36, wherein the experimentally-derived reference enrichment signature is generated from at least one biopanning experiment comprising at least one reference anti-ligand.
38. The method according to any one of paragraphs 35 to 37, wherein the in silico-derived reference enrichment signature is for an anti-ligand to a high expressed ligand of a target cell, tissue, or sample of interest, for an anti-ligand to an intermediary expressed ligand of a target cell, tissue, or sample of interest; or for an anti-ligand to a low expressed ligand of a target cell, tissue, or sample of interest, and/or
   wherein the experimentally-derived reference enrichment signature is for an anti-ligand to a high expressed ligand of a target cell, tissue, or sample of interest, for an anti-ligand to an intermediary expressed ligand of a target cell, tissue, or sample of interest; or for an anti-ligand to a low expressed ligand of a target cell, tissue, or sample of interest.
39. The method of any one of paragraphs 28 to 38, wherein the one or more discovery enrichment signature provided in step (b) is derived from a previously conducted anti-ligand library selection experiment.
40. The method of any one of paragraphs 28 to 39, wherein the at least one differentially expressed ligand is:
   a high expressed ligand of a target cell, tissue, or sample of interest;
   an intermediary expressed ligand of a target cell, tissue, or sample of interest; or,
   a low expressed ligand of a target cell, tissue, or sample of interest;
41. The method of any one of paragraphs 28 to 40, wherein the library of anti-ligands is a display library comprising a plurality of library members which display anti-ligands; optionally wherein the library is a phage display library and/or wherein the library is constructed from at least one of antibodies, antigen binding variants, derivatives and/or fragments thereof; scaffold molecules with engineered variable surfaces; receptors; and enzymes.
42. The method of any one of paragraphs 28 to 41, wherein the library of anti-ligands is a pool of anti-ligands generated by a previously conducted anti-ligand library selection experiment.
43. The method of any one of paragraphs 28 to 42, wherein the library of anti-ligands comprises at least one anti-ligand having a known copy number, optionally wherein the at least one anti-ligand having a known copy number is added to the library of anti-ligands at a known copy number.
44. The method of any one of paragraphs 28 to 43, wherein the ligand is at least one selected from antigens; receptor ligands; and enzyme targets that comprise at least one of carbohydrate; protein; peptide; lipid; polynucleotide; inorganic molecules and conjugated molecules; optionally wherein the ligand is a cell surface receptor, optionally wherein the cell surface receptor is in its native form.
45. An enrichment signature for an anti-ligand, wherein the enrichment signature is generated by step (c) of the method according to any one of paragraphs 1 to 27.
46. A method of preparing a pharmaceutical composition, the method comprising the step of adding an anti-ligand isolated by the method according to any one of paragraphs 1 to 44 to to a pharmaceutically acceptable carrier.
47. A pharmaceutical composition prepared by the method according to paragraph 46.
48. A pharmaceutical composition prepared by the method according to paragraph 47 for use in medicine.

1. A method of isolating from a library of anti-ligands at least one anti-ligand to at least one differentially-expressed target ligand in a target cell, tissue, or sample of interest, wherein the method comprises the steps of:
(a) providing one or more reference enrichment signature for the library of anti-ligands used;
(b) performing one or more round of differential biopanning on the library of anti-ligands to produce anti-ligand pools;
(c) performing high throughput sequencing on the anti-ligand pools produced during step (b), so as to generate a discovery enrichment signature for each anti-ligand in the anti-ligand pools;
(d) matching the one or more reference enrichment signature provided in step (a) with a discovery enrichment signature for an anti-ligand generated in step (c), so as to isolate at least one anti-ligand to at least one differentially-expressed target ligand in a target cell, tissue, or sample of interest.

2-32. (canceled)

33. The method according to claim 1, wherein the one or more reference enrichment signature provided in step (a) is an in silico-derived reference enrichment.

34. The method according to claim 1, wherein the one or more reference enrichment signature provided in step (a) is an experimentally-derived reference enrichment signature.

35. The method according to claim 1, wherein in step (a) two or more reference enrichment signatures are provided, wherein one or more of the reference enrichment signatures is an in silico-derived reference enrichment signature, and wherein one or more of the reference enrichment signatures is an experimentally-derived enrichment signature.

36. The method according to claim 35, wherein the in silico-derived reference enrichment signature is for an anti-ligand to a high expressed ligand of a target cell, tissue, or sample of interest, for an anti-ligand to an intermediary expressed ligand of a target cell, tissue, or sample of interest; or for an anti-ligand to a low expressed ligand of a target cell, tissue, or sample of interest, and/or wherein the experimentally-derived reference enrichment signature is for an anti-ligand to a high expressed ligand of a target cell, tissue, or sample of interest, for an anti-ligand to an intermediary expressed ligand of a target cell, tissue, or sample of interest; or for an anti-ligand to a low expressed ligand of a target cell, tissue, or sample of interest.

37. The method according to claim 1, wherein biopanning step (a) comprises the sub-steps of:
(i) providing a library of anti-ligands;
(ii) providing a first population of ligands comprising a ligand fixed to or incorporated in a subtractor ligand construct;
(iii) providing a second population of ligands comprising a ligand fixed to or incorporated in a target ligand construct;
(iv) determining an amount of the subtractor ligand construct and the target ligand construct in the populations using one or more equations derived from the universal law of mass action
$$\frac{{{[ C ]}^{c} [ D ]}^{d}}{{{[ A ]}^{a} [ B ]}^{b}} = {K}_{eq} ,$$
where:
A, B, C & D=are the participants in the reaction (reactants and products)
a, b, c, & d=the coefficients necessary for a balanced chemical equation so as to permit isolation of anti-ligand to differentially expressed target ligand;
(v) providing the amount of subtractor ligand construct as determined in step (iv);
(vi) providing the amount of target ligand construct as determined in step (iv);
(vii) providing separation means for isolating anti-ligand bound to the target ligand construct from anti-ligand bound to a subtractor ligand construct;
(viii) exposing the library of (i) to the ligand constructs provided by (v) and (vi) to permit binding of anti-ligands to ligands; and
(ix) using the separation means to isolate anti-ligand bound to the ligand fixed to or incorporated in the target ligand construct.

38. The method according to claim 1, wherein step (b) comprises performing two or more rounds of biopanning, three or more rounds of biopanning, or four or more rounds of biopanning.

39. The method according to claim 1, wherein the method further comprises a step of releasing the anti-ligand from the ligand.

40. A method of isolating from a library of anti-ligands at least one anti-ligand to at least one differentially-expressed target ligand in a target cell, tissue, or sample of interest, wherein the method comprises the steps of:
(a) providing one or more reference enrichment signature for an anti-ligand present in the library of anti-ligands used;
(b) providing one or more discovery enrichment signature for an anti-ligand present in the library of anti-ligands used; and
(c) matching the one or more reference enrichment signature provided in step (a) with the one or more discovery enrichment signature provided in step (b), so as to isolate at least one anti-ligand to at least one differentially-expressed target ligand in a target cell, tissue, or sample of interest.

41. The method according to claim 40, wherein the one or more reference enrichment signature provided in step (a) is an in silico-derived reference enrichment.

42. The method according to claim 40, wherein the one or more reference enrichment signature provided in step (a) is an experimentally-derived reference enrichment signature.

43. The method according to claim 40, wherein in step (a) two or more reference enrichment signatures are provided, wherein one or more of the reference enrichment signatures is an in silico-derived reference enrichment signature, and wherein one or more of the reference enrichment signatures is an experimentally-derived enrichment signature.

44. The method of claim 40, wherein the one or more discovery enrichment signature provided in step (b) is derived from a previously conducted anti-ligand library selection experiment.

45. An enrichment signature for an anti-ligand, wherein the enrichment signature is generated by step (c) of the method according to claim 1.

46. A method of preparing a pharmaceutical composition, the method comprising the step of combining:
an anti-ligand isolated by the method according to claim 1; and
a pharmaceutically acceptable carrier.

47. A pharmaceutical composition prepared by the method according to claim 46.

48. The method of claim 33, wherein the in silico-derived reference enrichment signature is generated using an equation derived from the universal law of mass of action, optionally wherein the in silico-derived signature is generated using the equation:
$${FA}_{T} = \frac{{rA}_{T}}{( {\sum }_{1}^{n} {rA}_{T} )} \times HR$$
where
FA_{T}=frequency of recovered antibody
rA_{T}=number of recovered antibodies
$${FA}_{T} = \frac{{rA}_{T}}{( {\sum }_{1}^{n} {rA}_{T} )} \times HR$$
HR=hit-rate, fraction of antibodies specific for the target cells, tissues, or experimental samples.

49. The method of claim 41, wherein the in silico-derived reference enrichment signature is generated using an equation derived from the universal law of mass of action, optionally wherein the in silico-derived signature is generated using the equation:
$${\sum }_{1}^{n} {rA}_{T} = Total number of recovered antibodies$$
where
FA_{T}=frequency of recovered antibody
rA_{T}=number of recovered antibodies
$${\sum }_{1}^{n} {rA}_{T} = Total number of recovered antibodies$$
HR=hit-rate, fraction of antibodies specific for the target cells, tissues, or experimental samples.

50. A method of preparing a pharmaceutical composition, the method comprising the step of combining:
an anti-ligand isolated by the method according to claim 40; and
a pharmaceutically acceptable carrier.

51. A pharmaceutical composition prepared by the method according to claim 50.
